(19) United States
(12) Patent Application Publication
(10) Pub. No.: US 2024/0264176 A1
(43) Pub. Date: Aug. 8, 2024

(54) METHODS AND MATERIALS FOR DETECTING PRION DISEASES

(71) Applicants: Regents of the University of Minnesota, Minneapolis, MN (US); Creighton University, Omaha, NE (US)

(72) Inventors: Peter A. Larsen, Minneapolis, MN (US); Tiffany M. Wolf, Minneapolis, MN (US); Marc D. Schwabenlander, Minneapolis, MN (US); Jason C. Bartz, Omaha, NE (US); Qi Yuan, Omaha, NE (US)

(21) Appl. No.: 18/567,172

(22) PCT Filed: Jun. 6, 2022
(86) PCT No.: PCT/US2022/032299

§ 371 Date: Dec. 5, 2023

Related U.S. Application Data
(60) Provisional application No. 63/197,822, filed on Jun. 7, 2021.

(51) Int. Cl. G01N 33/68; G01N 1/40

(52) U.S. Cl. CPC G01N 33/6896; G01N 1/4077; G01N 2001/4094; G01N 2800/2828

(57) ABSTRACT

This document provides methods and materials for detecting misfolded poly peptides. For example, devices (e.g., feeding devices) designed to collect samples from one or more mammals (e.g., one or more cervids) are provided. In some cases, methods and materials that can be used to detect one or more prion diseases (e.g., chronic wasting disease (CWD)) in a population of mammals (e.g., a herd of cervids) based, at least in part, on the presence or absence of misfolded polypeptides in a sample from one or more mammals that was obtained from a feeding device are provided.

### CROSS-REFERENCE TO RELATED APPLICATIONS

This application claims the benefit of U.S. Patent Application Ser. No. 63/197,822, filed on Jun. 7, 2021. The disclosure of the prior application is considered part of (and is incorporated by reference in) the disclosure of this application.

### STATEMENT REGARDING FEDERAL FUNDING

This invention was made with government support under AI077774 awarded by the National Institutes of Health, and with government support under CBET-1149424 awarded by the National Science Foundation. The government has certain rights in the invention.

### BACKGROUND

### 1. Technical Field

This document relates to methods and materials for detecting misfolded polypeptides. For example, this document provides devices (e.g., feeding devices) designed to collect samples from one or more mammals (e.g., one or more cervids). In some cases, methods and materials provided herein can be used to assess the presence or absence of one or more prion diseases (e.g., chronic wasting disease (CWD)) in a population of mammals (e.g., a herd of cervids) based, at least in part, on the presence or absence of one or more misfolded polypeptides in a sample from one or more mammals that was obtained from a feeding device provided herein.

### 2. Background Information

CWD in an emerging prion disease in North America and Europe. CWD has been detected in 30 states (USGS National Wildlife Health Center, “Distribution of Chronic Wasting Disease in North America,” usgs.gov/centers/nwhc/science/expanding-distribution-chronic-wasting-disease?qt-science_center_objects=0#qt-science_center_objects) in the United States and three Canadian provinces (Canadian Food Inspection Agency, “Herds infected with chronic wasting disease in Canada,” inspection.canada.ca/animal-health/terrestrial-animals/diseases/reportable/cwd/herds-infected/eng/1554298564449/1554298564710). The recent discovery of CWD in reindeer and moose in Norway, Finland, and Sweden (Benestad et al., Vet. Res., 47:88 (2016); and Osterholm et al., mBio, 10(4):e01091-19 (2019)) has resulted in growing concern of CWD transmission and emergence worldwide. In CWD endemic areas, the disease incidence in free-ranging deer herds can be greater than 50% and can negatively impact cervid populations (Miller et al., J. Wildl. Dis., 40(2):320-327 (2004)). The zoonotic potential of CWD is poorly understood; however, there is evidence for the potential of CWD to transmit to sympatric species such as cattle, cats, swine sheep and, as suggested recently, humans (Wolfe et al., J. Wildl. Dis., 58(1):40-49 (2021); Hamir et al., Vet. Pathol., 44:487-493 (2007); Hamir et al., J. Vet. Diagn. Invest., 18:558-565 (2006); Hamir et al., J. Vet. Diagn. Invest., 17:276-281 (2005); Bartz et al., Virology, 251:297-301 (1998); Moore et al., J. Virology, 91:e00926-00917 (2017); and Hannaoui et al., bioRxiv, 2022.2004.2019.488833, doi: 10.1101/2022.04.19.488833 (2022)).

### SUMMARY

Prion diseases impact a wide range of host species that include humans (Creutzfeldt-Jakob disease, CJD), cattle (bovine spongiform encephalopathy, BSE or “mad cow disease”), cervids (CWD), dromedary camels, and sheep and goats (scrapie). Prions are comprised of a misfolded self-propagating form (Prp^{Sc}) of a normal cellular prion polypeptide (PrP^{C}). Currently, the most widely accepted way to diagnose prion diseases such as CWD is by post-mortem examination of brain and lymphoid tissues for the presence of Prp^{Sc}.

This document provides methods and materials for detecting misfolded polypeptides (e.g., Prp^{Sc}). For example, this document provides devices (e.g., feeding devices having at least one sample collection surface) designed to collect samples (e.g., biological samples such as saliva and/or mucus) from one or more mammals (e.g., one or more non-human mammal such as cervids). One or more samples deposited on a device provided herein can be assessed for the presence or absence of one or more misfolded polypeptides (e.g., Prp^{Sc}) present in the sample. In some cases, methods and materials provided herein can be used to detect and, optionally, monitor, one or more prion diseases (e.g., CWD) in a population of mammals (e.g., a herd of cervids) based, at least in part, on the presence or absence of one or more misfolded polypeptides (e.g., Prp^{Sc}) in a sample from one or more mammals that was obtained from a device provided herein.

As demonstrated herein, misfolded polypeptides (e.g., Prp^{Sc}) can be recovered from sample collection surfaces using swabs and can be identified. For example, feeding devices having at least one sample collection surface can be placed in specific geographic regions and/or farms such that when mammals (e.g., cervids) feed from the feeding devices saliva and/or mucus is left on the sample collection surface, samples can be obtained (e.g., via swab) from the sample collection surface, and the samples can be assessed for the presence or absence of one or more misfolded polypeptides (e.g., Prp^{Sc}). The presence of one or more misfolded polypeptides (e.g., Prp^{Sc}) can be used to detect the presence of one or more prion diseases in a population of mammals (e.g., a herd of cervids).

Having the ability to detect one or more prion diseases in a population of mammals (e.g., a herd of cervids) as described herein (e.g., by determining the presence of one or more misfolded polypeptides (e.g., Prp^{Sc}) deposited on a sample collection surface of a feeding device provided herein) provides a unique and unrealized opportunity for herd-level environmental surveillance of prion diseases. For example, herd-level surveillance of prion diseases in wild and domestic populations of mammals (e.g., herds of cervids) can be used to monitor and, optionally, control disease transmission.

In general, one aspect of this document features methods for assessing a population of non-human mammals for a prion disease. The methods can include, or consist essentially of, swabbing a sample collection surface of a feeding device to obtain a swab comprising a sample, where the feeding device includes a reservoir and a sample collection surface, where the reservoir contains food, and where the food can pass from the reservoir to the sample collection surface; extracting polypeptides from the sample to obtain an extract; concentrating the extract to obtain a concentrated extract; detecting a presence of a misfolded polypeptide in the concentrated extract; identifying the population of non-human mammals as having the prion disease if the presence of the misfolded polypeptide is detected; and identifying the population of non-human mammals as not having the prion disease if the presence of the misfolded polypeptide is not detected. The sample collection surface can be stainless steel, mica, slate, aluminum, ceramic, or glass. The food can be corn, soybeans, oats, or commercial feed pellets. The sample can be saliva, mucus, or tongue epithelial cells. The population of non-human mammals can include elk, fallow deer, marsh deer, mule deer, muntjac, moose, pampas deer, red deer, reindeer, roe deer, sambar deer, sika, white-tailed deer, antelope, goats, camels, mink, cats, cows, sheep, mice, rats, hamsters, brocket, chital, macaques, lemurs, spider monkeys, or chimpanzees. The feeding device can be located in a wilderness area. The feeding device can be located in an urban setting, a suburban setting, or a rural setting. The feeding device can be unattended by a human for from about 1 to about 14 days. The feeding device can be located on a farm. The sample can be obtained daily. The sample can be obtained weekly. The swab can be a cotton swab or a foam swab. The swab can be stored in a solution prior to extracting polypeptides from the sample to obtain the extract. The solution can be a phosphate-buffered saline (PBS) solution. The swab can be stored at a temperature of from about −80° C. to about 4° C. The extracting step can include sonication. The concentrating step can include vacuum concentration. The misfolded polypeptide can be detected using real-time quaking-induced conversion (RT-QuIC), enzyme-linked immunosorbent assay (ELISA), immunohistochemistry (IHC), protein misfolding cyclic amplification (PMCA), or western blotting. The misfolded polypeptide can be a misfolded polypeptide associated with scrapie (Prp^{Sc}), a misfolded polypeptide associated with chronic wasting disease (PrP^{CWD}), a misfolded polypeptide associated with bovine spongiform encephalopathy (PrP^{BSE}), a misfolded polypeptide associated with Creutzfeldt-Jakob Disease (PrP^{CJD}), a misfolded polypeptide associated with feline spongiform encephalopathy (PrP^{FSE}), a misfolded polypeptide associated with transmissible mink encephalopathy (PrP^{TME}), or a misfolded polypeptide associated with camel spongiform encephalopathy (PrP^{CSE}). The prion disease can be chronic wasting disease (CWD), transmissible mink encephalopathy (TME), bovine spongiform encephalopathy (BSE), scrapie, feline spongiform encephalopathy, ungulate spongiform encephalopathy, or camilid spongiform encephalopathy.

In another aspect, this document features methods for detecting a misfolded polypeptide. The methods can include, or consist essentially of, swabbing a sample collection surface to obtain a swab comprising a sample; extracting polypeptides from the sample to obtain an extract; concentrating the extract to obtain a concentrated extract; and detecting a presence of the misfolded polypeptide in the concentrated extract. The sample collection surface can be stainless steel, mica, slate, aluminum, ceramic, or glass. The collection surface can be in a food processing facility, a water-treatment facility, or a hospital. The sample can be blood, urine, feces, saliva, or mucus. The sample can be obtained daily. The sample can be obtained weekly. The swab can be a cotton swab or a foam swab. The swab can be stored in a solution prior to extracting polypeptides from the sample to obtain the extract. The solution can be a PBS solution. The swab can be stored at a temperature of from about −80° C. to about 4° C. The extracting can include sonication. The concentrating step can include vacuum concentration. The misfolded polypeptide can be detected using RT-QuIC, ELISA, IHC, PMCA, or western blotting. The misfolded polypeptide can be a misfolded tau polypeptide, a misfolded alpha-synuclein polypeptide, or a misfolded amyloid beta polypeptide. The misfolded polypeptide can be associated with a prion disease. The prion disease can be Creutzfeldt-Jakob disease (CJD), Gerstmann-Straussler-Scheinker disease (GSS), fatal familial insomnia (FFI), Alzheimer's disease, or Parkinson's disease.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1H. Recovery of a hyper (HY) strain of transmissible mink encephalopathy (TME) Prp^{Sc} from foam or cotton swabs using shaking. Representative 96-well immunoblots (FIGS. 1A, 1C, 1E, and 1G) and quantification (FIGS. 1B, 1D, 1F, and 1H column graph) of recovered HY Prp^{Sc} from three sequential extractions (1^{st}, 2^{nd}, 3^{rd}) of both foam and cotton swabs dried at room temperature for different lengths of time. Line graphs represent the moisture content of swabs wetted with HY TME brain homogenate (solid line) or ultrapure water (dashed line) after drying at room temperature. Results of Prp^{Sc} recovery are expressed as mean of total recovery ±SEM, n=4; data points in the line graphs represent the mean value of moisture content, n=3. * indicates significant difference (P<0.05) between the sample and respective undried control (0 hours).

FIGS. 2A-2D. Recovery of HY TME Prp^{Sc} extracted from foam swabs extracted using sonication. Representative 96-well immunoblots (FIGS. 2A and 2C) and quantification (FIGS. 2B and 2D) of recovered HY TME Prp^{Sc} from three sequential extractions (1^{st}, 2^{nd}, 3^{rd}) of foam swabs either undried (0 hours) or dried for 24 hours at room temperature. Sonication times consist of 1, 3, 6, or 12 cycle(s) of pulse treatment with 5 seconds of sonication followed by 5 seconds of incubation. Results of prion recovery are expressed as mean of total recovery ±SEM, n=4.

FIGS. 3A-3F. Recovery of CWD Prp^{Sc} extracted from foam swabs with sonication. Representative 96-well immunoblots (FIGS. 3A, 3C, and 3E) and quantification (FIGS. 3B, 3D, and 3F) of recovered CWD Prp^{Sc} from three sequential extractions (1^{st}, 2^{nd}, 3^{rd}) of foam swabs either undried (0 hours) or dried for 24 hours at room temperature. 15 seconds (3 cycles of pulse treatment) of sonication was used for each extraction. Results of prion recovery are expressed as mean of total recovery ±SEM, n=4. * indicates significant difference (P<0.05) between the 24-hour dried sample and respective undried control (0 hours).

FIGS. 4A-4L. Recovery of CWD Prp^{Sc} from glass, stainless steel, or wood. Representative 96-well immunoblots (FIGS. 4A-4C) and quantification (FIGS. 4D-4L) of recovered CWD Prp^{Sc} from foam swabs (combined extract of two sequential extractions for each swab) applied to either undried (control) or 24 hour-dried surfaces at room temperature. 15 seconds (3 cycles of pulse treatment) of sonication was used for each extraction. For controls, CWD Prp^{Sc} at the highest contamination levels were applied. Results of prion recovery are expressed as mean of total recovery ±SEM, n=3. * indicates significant difference (p<0.05) between the 24-hour dried sample and respective undried control (0 hours).

FIGS. 5A-5D. Prion recovery from swab-free processes under varied treatment conditions. HY or CWD Prp^{Sc} spiked into 200 μL, 400 μL, or 600 μL of DPBS buffer were subjected to 15 seconds, 30 seconds, or 60 seconds (consisting of 3, 6, or 12 cycles of 5 seconds sonication followed with 5 seconds incubation) of sonication. Samples were then vacuum-concentrated for 3 hours, 4 hours, or 5 hours. For testing the impact of sonication duration, the buffer volume was fixed at 200 μL and lyophilization time was fixed at 3 hours. For testing the impact of buffer volume, the sonication time was fixed at 15 seconds and vacuum-concentration time was fixed at 5 hours. For testing the impact of concentration duration, the sonication time was fixed at 15 seconds and the buffer volume was fixed at 200 μL. * indicates significant difference (p<0.05, n=4) compared to the control. # indicates significant difference (p<0.05, n=4) compared to the respective controls (i.e., 15 seconds, 200 μL, or 3 hours).

FIGS. 6A-6C. An exemplary feeding device having a sample collection surface. FIG. 6A. A side view of a pole-mounted feeding device for surveillance of misfolded polypeptides (e.g., Prp^{Sc}). FIG. 6B. An expanded view of the boxed area of FIG. 6A. Grain can flow from a central reservoir and can pass through a hinged stainless steel plate and onto a stainless steel sample collection surface. FIG. 6C. A schematic image of a swab collecting a sample of CWD prions from the stainless steel surface of the feeding device and transferring the sample to a tube (left panel) and using real-time quaking-induced conversion (RT-QuIC) to identify the presence of misfolded polypeptides (e.g., Prp^{Sc}) in the sample (right panel).

FIGS. 7A-7B. An exemplary feeding device having multiple sample collection surfaces. FIG. 7A. A side view of a feed-bunk for surveillance of misfolded polypeptides (e.g., Prp^{Sc}). Grain can flow from a central reservoir through release spouts and onto stainless steel sample collection surfaces. FIG. 7B. A top view of the feed-bunk shown in FIG. 7A. A central grain reservoir can include four release spouts, each of which release grain onto a separate stainless steel sample collection surface.

FIGS. 8A-8C. RT-QuIC detection of CWD prion from swabs and stainless steel—1^{st} extraction as maxpoint ratio. FIG. 8A. RT-QuIC detection methodology for CWD Prp^{Sc}. FIG. 8B. RT-QuIC detection for serial 10-fold dilutions of CWD Prp^{Sc} directly applied to swabs that were extracted immediately. FIG. 8C. RT-QuIC detection for serial 10-fold dilutions of CWD Prp^{Sc} applied to a stainless steel surface that was swabbed after 24-hour drying at 22° C. followed with immediate swab extraction. Swab extract was analyzed for the presence of Prp^{Sc} capable of misfolding recHaPrP to amyloids in the RT-QuIC reaction, and was expressed as the maxpoint ratio (MPR, mean±standard deviation) which was determined as the ratio of maximum thioflavin T (ThT) fluorescence in the entire RT-QuIC run to the ThT fluorescence of the starting cycle of RT-QuIC reaction. The threshold of a positive signal was set at 2 (dashed line).

FIGS. 9A-9B. RT-QuIC detection of CWD prion from stainless steel—2^{nd} extraction as maxpoint ratio. RT-QuIC detection methodology for CWD Prp^{Sc} is as shown in FIG. 8A. FIG. 9A. RT-QuIC detection for serial 10-fold dilutions of CWD Prp^{Sc} directly applied to swabs that were extracted immediately. FIG. 9B. RT-QuIC detection for serial 10-fold dilutions of CWD Prp^{Sc} applied to a stainless steel surface that was swabbed after 24-hour drying at 22° C. followed with immediate swab extraction. Swab extract was analyzed for the presence of Prp^{Sc} capable of misfolding recHaPrP to amyloids in the RT-QuIC reaction, and was expressed as the MPR (mean±standard deviation) which was determined as the ratio of maximum ThT fluorescence in the entire RT-QuIC run to the ThT fluorescence of the starting cycle of RT-QuIC reaction. The threshold of a positive signal was set at 2 (dashed line).

FIGS. 10A-10B. RT-QuIC detection of CWD prion from swabs and stainless steel—1^{st} extraction as rate of amyloid formation. RT-QuIC detection methodology for CWD Prp^{Sc} is as shown in FIG. 8A. FIG. 10A. RT-QuIC detection for serial 10-fold dilutions of CWD Prp^{Sc} directly applied to swabs that were extracted immediately. FIG. 10B. RT-QuIC detection for serial 10-fold dilutions of CWD Prp^{Sc} applied to a stainless steel surface that was swabbed after 24-hour drying at 22° C. followed with immediate swab extraction. Swab extract was analyzed for the presence of Prp^{Sc} capable of misfolding recHaPrP to amyloids in the RT-QuIC reaction, and was expressed as rate of amyloid formation (RAF, mean±standard deviation) which was determined as the inverse of RT-QuIC reaction time to reach the fluorescent threshold (MPR=2).

FIGS. 11A-11B. RT-QuIC detection of CWD prion from swabs and stainless steel—2^{nd} extraction as rate of amyloid formation. RT-QuIC detection methodology for CWD Prp^{Sc} is as shown in FIG. 8A. FIG. 11A. RT-QuIC detection for serial 10-fold dilutions of CWD Prp^{Sc} directly applied to swabs that were extracted immediately. FIG. 11B. RT-QuIC detection for serial 10-fold dilutions of CWD Prp^{Sc} applied to a stainless steel surface that was swabbed after 24-hour drying at 22° C. followed with immediate swab extraction. Swab extract was analyzed for the presence of Prp^{Sc} capable of misfolding recHaPrP to amyloids in the RT-QuIC reaction, and was expressed as RAF (mean±standard deviation) which was determined as the inverse of RT-QuIC reaction time to reach the fluorescent threshold (MPR=2).

FIGS. 12A-12E. RT-QuIC detection of samples recovered from DPBS-wetted swabs and uncontaminated stainless steel. FIG. 12A. RT-QuIC detection methodology for samples recovered from negative swab and surface controls. FIG. 12B and FIG. 12C. RT-QuIC detection of samples recovered from DPBS-wetted swabs that were extracted immediately. FIG. 12D and FIG. 12E. RT-QuIC detection of samples recovered from uncontaminated stainless steel by swabs with immediate extraction. Swab extract was analyzed for the presence of Prp^{Sc} capable of misfolding recHaPrP to amyloids in the RT-QuIC reaction, and was expressed as (1) MPR (mean±standard deviation) which was determined as the ratio of maximum ThT fluorescence in the entire RT-QuIC run to the ThT fluorescence of the starting cycle of RT-QuIC reaction (FIG. 12B and FIG. 12D); and (2) RAF (mean±standard deviation) which was determined as the inverse of RT-QuIC reaction time to reach the fluorescent threshold (MPR=2; FIG. 12C and FIG. 12E).

FIG. 13. RT-QuIC detection of misfolded polypeptides swabbed from stainless steel surface as maxpoint ratio (MPR). All misfolded polypeptides of interest diluted to the level specified in Table 5 were applied to stainless steel surface for 24-hour drying at room temperature followed with immediate swab extraction and concentration. Swab extract was analyzed for the presence of Prp^{Sc} capable of misfolding recHaPrP to amyloids in the RT-QuIC reaction and expressed as the maxpoint ratio (MPR, mean±standard deviation) which was determined as the ratio of maximum thioflavin T (ThT) fluorescence in the entire RT-QuIC run to the ThT fluorescence of the starting cycle of RT-QuIC reaction. The threshold of a positive signal was set at 2 (red dashed line). RT-QuIC detection of all samples were for their respective dilutions as listed in Table 5 except for scrapie at dilution of log −3. In each RT-QuIC plate, samples without misfolded proteins (Blank), normal prion protein (Negative), and/or known misfolded proteins (Positive) were tested as controls. Brain homogenates (all diluted to log −3, log −4 for cattle adapted TME) were directly added to RT-QuIC (307 CL, Scrapie, Cattle TME, TME, and squirrel monkey) as detection controls. Numbers from 1 to 27 were sample ID referring to samples listed in Table 5.

### DETAILED DESCRIPTION

This document provides methods and materials for detecting misfolded polypeptides (e.g., Prp^{Sc}). For example, this document provides devices (e.g., feeding devices having at least one sample collection surface) (e.g., biological samples such as saliva and/or mucus) from one or more (e.g., one or more non-human mammals such as cervids). In some cases, one or more samples from one or more mammals (e.g., one or more cervids) can be deposited on a sample collection surface of a device provided herein, and the sample(s) can be obtained from a sample collection surface and can be assessed for the presence or absence of one or more misfolded polypeptides (e.g., Prp^{Sc}) present in the sample. As described herein, misfolded polypeptides (e.g., Prp^{Sc}) can be recovered from sample collection surfaces and can be identified. For example, one or more feeding devices each having at least one sample collection surface can be placed in specific geographic regions and/or farms to collect saliva and/or mucus samples from a population of mammals (e.g., a herd of cervids) that feed from the feeding devices. This document also provides methods and materials to detect and, optionally, monitor, one or more prion diseases (e.g., CWD) in a population of mammals (e.g., a herd of cervids) based, at least in part, on the presence or absence of one or more misfolded polypeptides (e.g., Prp^{Sc}) in a sample from one or more mammals (e.g., one or more cervids) that was obtained from a device provided herein. For example, one or more samples from one or more mammals (e.g., one or more cervids) can be assessed for the presence or absence of one or more misfolded polypeptides (e.g., Prp^{Sc}). In some cases, the presence of one or more misfolded polypeptides (e.g., Prp^{Sc}) in a sample that was obtained from one or more mammals (e.g., one or more cervids) in a population of mammals (e.g., a herd of cervids) can be used to determine that at least one mammal (e.g., at least one cervid) in the population of mammals (e.g., the herd of cervids) has one or more prion diseases. In some cases, the absence of one or more misfolded polypeptides (e.g., Prp^{Sc}) in a sample that was obtained from one or more mammals (e.g., one or more cervids) in a population of mammals (e.g., a herd of cervids) can be used to determine that the population of mammals (e.g., the herd of cervids) does not have any prion diseases.

A device provided herein (e.g., a device such as a feeding device having at least one sample collection surface) can include any number of sample collection surface(s). In some cases, a feeding device provided herein can include at least one (e.g., one, two, three, four, five, or more) sample collection surface. For example, a feeding device provided herein can include from 1 to about 10 sample collection surfaces (e.g., from 1 to about 7, from 1 to about 5, from 1 to about 4, from 1 to about 3, from 2 to about 10, from 5 to about 10, from 1 to about 10, from 7 to about 10, from 2 to about 8, from 3 to about 5, from 2 to about 4, from 5 to about 7, or from 6 to about 8 sample collection surfaces). In some cases, a feeding device provided herein can include a plurality of (e.g., two, three, four, five, or more) sample collection surfaces. In some cases, a feeding device provided herein can include a single sample collection surface.

A sample collection surface of a device provided herein (e.g., a device such as a feeding device having at least one sample collection surface) can be made from any appropriate material. Examples of materials that can be used as a sample collection surface of a feeding device provided herein include, without limitation, stainless steel, mica, slate, aluminum, ceramic, and glass. In cases where a feeding device provided herein includes a plurality of sample collection surfaces, the sample collection surfaces can be made from the same material or can be made from different materials.

A device provided herein (e.g., a device such as a feeding device having at least one sample collection surface) can be placed in any appropriate environment. In cases where a device provided herein is feeding device, the feeding device can be placed where one or more mammals (e.g., one or more non-human such as cervids) can access the feeding device. Examples of environments in which a device provided herein can be placed include, without limitation, environments where domestic (e.g., livestock) herds live (e.g., farms, ranches, fenced hunting preserves, and zoos, environments where wild herds live (e.g., forests, grasslands, of agricultural fields such as alfalfa, corn, soybeans, potatoes, wheat, and barley fields), environments where human live (e.g., rural areas, suburban areas, and urban areas), food processing facilities (e.g., meat processing facilities), water-treatment facilities, and hospitals (e.g., human hospitals and veterinary hospitals).

When a device provided herein (e.g., a device such as a feeding device having at least one sample collection surface) is a feeding device, the feeding device can provide any appropriate type of food. In some cases, a feeding device provided herein can be used to provide grains. In some cases, a feeding device provided herein can be used to provide pellets. Examples of foods that can be provided using a feeding device provided herein include, without limitation, corn, soybeans, oats, commercial feed pellets (e.g., commercial deer feed pellets), and any combinations thereof.

In some cases, a feeding device provided herein (e.g., a feeding device having at least one sample collection surface) can include a reservoir (e.g., a food reservoir). For example, a reservoir can be used to store and deliver food such that the food passes across a sample collection surface of a feeding device provided herein. In some cases, food passing from a reservoir of a feeding device provided herein to a sample collection surface of a feeding device provided herein can pass through a spout. In some cases, food passing from a reservoir of a feeding device provided herein to a sample collection surface of a feeding device provided herein can pass through a plate (e.g., a hinged plate). For example, food passing from a reservoir of a feeding device provided herein to a sample collection surface of a feeding device provided herein can pass through a plate (e.g., a hinged plate) that can be a sample collection surface. In some cases, food can pass from a reservoir of a feeding device provided herein to a sample collection surface of a feeding device provided herein in such a manner as to increase interaction of one or more mammals (e.g., one or more non-human such as cervids) with the sample collection surface. For example, food can pass from a reservoir of a feeding device provided herein and can accumulate at a point that is adjacent to a sample collection surface of a feeding device provided herein such that one or more mammals (e.g., one or more non-human such as cervids) leave sample on the sample collection surface while reaching across the sample collection surface to reach the food.

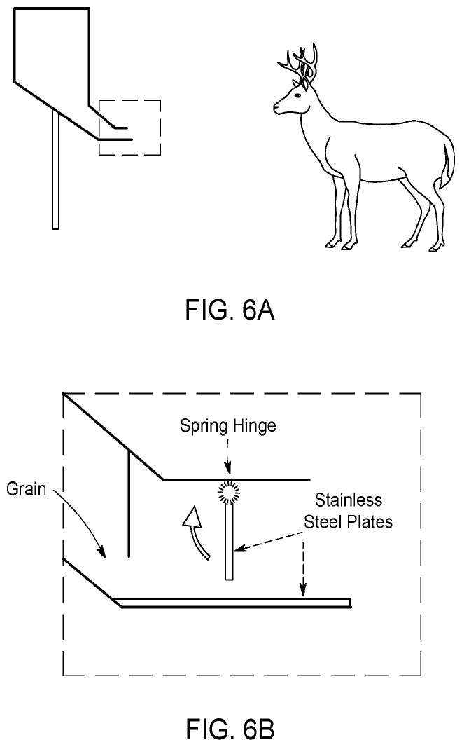

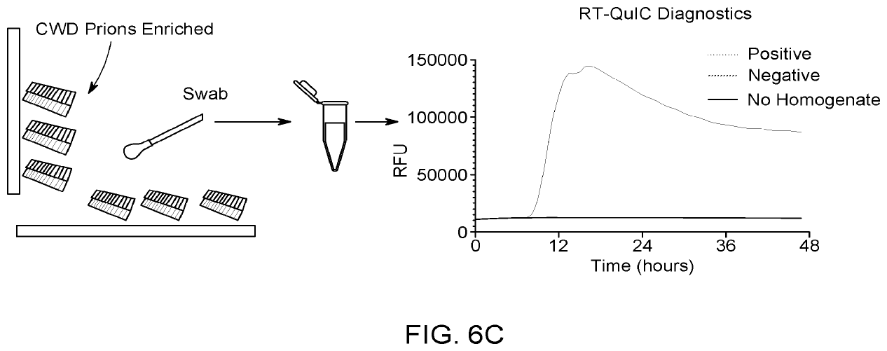

In some cases, a feeding device provided herein (e.g., a feeding device having at least one sample collection surface) can be as shown in FIG. 6.

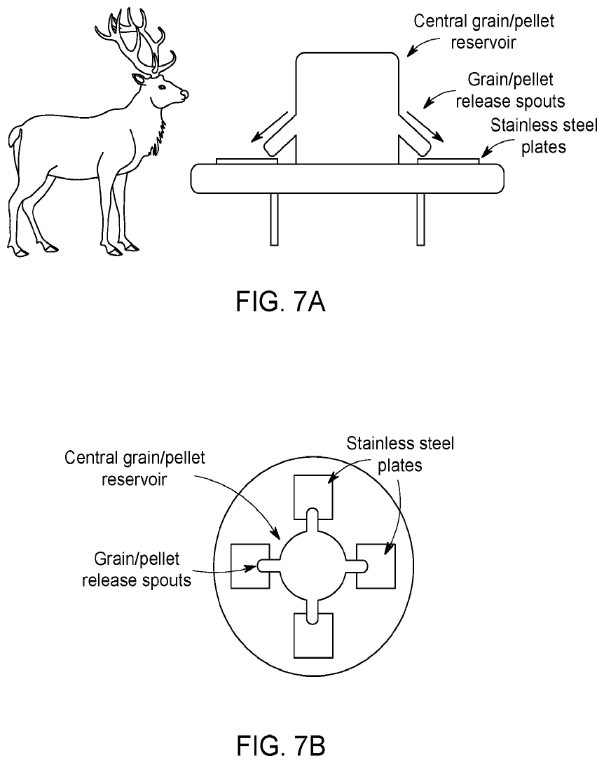

In some cases, a feeding device provided herein (e.g., a feeding device having at least one sample collection surface) can be as shown in FIG. 7.

A device provided herein (e.g., a device such as a feeding device having at least one sample collection surface) can be used to collect any appropriate type of sample. Examples of samples that can be obtained from a sample collection surface of a device provided herein include, without limitation, biological fluids (e.g., blood, urine, and feces), saliva, and mucus. When a device provided herein is a feeding device, the feeding device can be used to collect any appropriate type of sample from one or more mammals (e.g., one or more non-human such as cervids). In some cases, when a mammal (e.g., a cervid) feeds from a feeding device provided herein, the mammal will deposit one or more samples on the sample collection surface of the feeding device. Examples of samples that can be deposited on a sample collection surface of a feeding device provided herein by a mammal when it feeds from the feeding device include, without limitation, saliva, mucus, and epithelial cells (e.g., tongue epithelial cells).

When a device provided herein (e.g., a device such as a feeding device having at least one sample collection surface) is a feeding device, any appropriate mammal (e.g., any appropriate non-human mammal) can feed from the feeding device and can deposit one or more samples on a sample collection surface of the feeding device. In some cases, a non-human mammal can be a cervid (e.g., can be a member of the Cervidae family). In some cases, a non-human mammal can be a wild non-human mammal (e.g., wildlife). In some cases, a non-human mammal can be a domesticated non-human mammal (e.g., livestock). Examples of non-human mammals that can leave a sample on a feeding device provided herein include, without limitation, elk, fallow deer, marsh deer, mule deer, muntjac, moose, pampas deer, red deer, reindeer, roe deer, sambar deer, sika, white-tailed deer, antelope, goats, camels, mink, cats, cows, sheep, mice, rats, hamsters, brocket, chital, and non-human primates (e.g., macaques, lemurs, spider monkeys, and chimpanzees).

This document also provides methods for detecting the presence or absence of one or more misfolded polypeptides (e.g., Prp^{Sc}) in one or more samples from one or more mammals (e.g., one or more cervids) that were obtained from a device provided herein (e.g., a device such as a feeding device having at least one sample collection surface). For example, a sample from one or more mammals (e.g., one or more cervids) can be obtained from a sample collection surface of a feeding device provided herein, and can be assessed for the presence or absence of one or more misfolded polypeptides (e.g., Prp^{Sc}). In some cases, the presence of one or more misfolded polypeptides (e.g., Prp^{Sc}) in a sample from one or more mammals (e.g., one or more cervids) that was obtained from a sample collection surface of a feeding device provided herein can be used to identify a population of mammals (e.g., a herd of cervids) as having one or more prion diseases (e.g., based, at least in part, on the presence of the misfolded polypeptides such as Prp^{Sc}). For example, when one or more misfolded polypeptides (e.g., Prp^{Sc}) are detected in a sample that was obtained from a sample collection surface of a feeding device provided herein, a population of mammals (e.g., a herd of cervids) in the area where the feeding device is placed can be identified as having one or more prion diseases. In some cases, the absence of one or more misfolded polypeptides (e.g., Prp^{Sc}) in a sample from one or more mammals (e.g., one or more cervids) that was obtained from a sample collection surface of a feeding device provided herein can be used to identify a population of mammals (e.g., a herd of cervids) as not having one or more prion diseases (e.g., based, at least in part, on the absence of the misfolded polypeptides such as Prp^{Sc}). For example, when one or more misfolded polypeptides (e.g., Prp^{Sc}) are not detected in a sample that was obtained from a sample collection surface of a feeding device provided herein, a population of mammals (e.g., a herd of cervids) in the area where the feeding device is placed can be identified as not having one or more prion diseases.

Any appropriate method can be used to collect a sample from a sample collection surface of a device provided herein (e.g., a device such as a feeding device having at least one sample collection surface). In some cases, a swab can be used to collect a sample from a sample collection surface of a feeding device provided herein. Any appropriate swab can be used to collect a sample from a sample collection surface of a feeding device provided herein. Examples of swabs that can be used to collect a sample from a sample collection surface of a feeding device provided herein include, without limitation, cotton swabs (e.g., cotton-tipped swabs) and foam swabs (e.g., foam-tipped swabs).

A sample san be obtained from a sample collection surface of a device provided herein (e.g., a device such as a feeding device having at least one sample collection surface) at any appropriate time. In cases where a feeding device provided herein is located in a wilderness area (e.g., in a forest or grassland), agricultural field, urban, suburban, or rural setting), a sample can be obtained after the feeding device is left unattended by humans for longer than about 1 day. For example, a sample can be obtained from a feeding device provided herein that is located in a wilderness area from about 1 day to about 14 days after being left unattended by humans. In cases where a feeding device provided herein is located in an urban, suburban, or rural setting, a sample can be obtained after the feeding device is left unattended by humans for longer than about 1 day. For example, a sample can be obtained from a feeding device provided herein that is located in an urban, suburban, or rural setting from about 1 day to about 14 days after being left unattended by humans. In cases where a feeding device provided herein is located on a farm, a sample can be obtained every day. In cases where a feeding device provided herein is located on a farm, a sample can be obtained every week.

In some cases, a sample that was obtained from a sample collection surface of a device provided herein (e.g., a device such as a feeding device having at least one sample collection surface) can be extracted from a swab used to obtain the sample. Any appropriate method can be used to extract a sample from a swab. In some cases, shaking can be used to extract a sample from a swab. For example, a swab used to obtain a sample from the sample collection surface of a feeding device provided herein can be shaken (e.g., can be shaken in a buffer solution such as a PBS solution) on a micro tube mixer (Tomy MT-360, speed 5). A swab used to obtain a sample from the sample collection surface of a feeding device provided herein can be shaken for any amount of time. For example, a swab used to obtain a sample from the sample collection surface of a feeding device provided herein can be shaken for from about 5 minutes to about 40 minutes (e.g., from about 5 minutes to about 30 minutes, from about 5 minutes to about 20 minutes, from about 5 minutes to about 10 minutes, from about 10 minutes to about 40 minutes, from about 20 minutes to about 40 minutes, from about 30 minutes to about 40 minutes, from about 10 minutes to about 30 minutes, from about 15 minutes to about 25 minutes, from about 10 minutes to about 20 minutes, or from about 20 minutes to about 30 minutes). In some cases, a swab used to obtain a sample from the sample collection surface of a feeding device provided herein can be shaken for about 30 minutes. A shaking step can be performed any number of times. For example, a swab used to obtain a sample from the sample collection surface of a feeding device provided herein can be shaken from about 1 time to about 5 times (e.g., about 3 times). A shaking step can be performed at any temperature. In some cases, a swab used to obtain a sample from the sample collection surface of a feeding device provided herein can be shaken at room temperature.

In some cases, sonication can be used to extract a sample from a swab used to obtain a sample from the sample collection surface of a device provided herein (e.g., a device such as a feeding device having at least one sample collection surface). For example, a swab used to obtain a sample from the sample collection surface of a feeding device provided herein can be sonicated (e.g., can be sonicated in a buffer solution such as a PBS solution) at any appropriate speed (e.g., about speed 5 on a Tomy MT-360). A swab used to obtain a sample from the sample collection surface of a feeding device provided herein can be sonicated at any appropriate amplitude. For example, a swab used to obtain a sample from the sample collection surface of a feeding device provided herein can be sonicated at from about amplitude 15 to about amplitude 20 (e.g., about amplitude 17). For example, a swab used to obtain a sample from the sample collection surface of a feeding device provided herein can be sonicated with an ultrasonic intensity of from about 0.7 Watts/cm² to about 7 Watts/cm² (e.g., from about 0.7 Watts/cm² to about 6 Watts/cm², from about 0.7 Watts/cm² to about 5 Watts/cm², from about 0.7 Watts/cm² to about 4 Watts/cm², from about 0.7 Watts/cm² to about 3 Watts/cm², from about 0.7 Watts/cm² to about 2 Watts/cm², from about 0.7 Watts/cm² to about 1 Watts/cm², from about 1 Watts/cm² to about 7 Watts/cm², from about 2 Watts/cm² to about 7 Watts/cm², from about 3 Watts/cm² to about 7 Watts/cm², from about 4 Watts/cm² to about 7 Watts/cm², from about 5 Watts/cm² to about 7 Watts/cm², from about 6 Watts/cm² to about 7 Watts/cm², from about 1 Watts/cm² to about 6 Watts/cm², from about 2 Watts/cm² to about 5 Watts/cm², from about 3 Watts/cm² to about 4 Watts/cm², from about 1 Watts/cm² to about 3 Watts/cm², from about 2 Watts/cm² to about 4 Watts/cm², from about 3 Watts/cm² to about 5 Watts/cm², or from about 4 Watts/cm² to about 6 Watts/cm²). A swab used to obtain a sample from the sample collection surface of a feeding device provided herein can be sonicated for any amount of time. For example, a swab used to obtain a sample from the sample collection surface of a feeding device provided herein can be sonicated for from about 15 seconds to about 60 seconds (e.g., from about 15 seconds to about 60 seconds, from about 15 seconds to about 45 seconds, from about 15 seconds to about 30 seconds, from about 30 seconds to about 60 seconds, from about 45 seconds to about 60 seconds, from about 20 seconds to about 40 seconds, from about 20 seconds to about 30 seconds, from about 30 seconds to about 40 seconds, or from about 40 seconds to about 50 seconds). In some cases, a swab used to obtain a sample from the sample collection surface of a feeding device provided herein can be sonicated for about 15 seconds. A sonication step can be performed any number of times. For example, a swab used to obtain a sample from the sample collection surface of a feeding device provided herein can be sonicated from about 1 time to about 12 times. In some cases, a sonication step can be performed 1, 3, 6, or 12 times. A sonication step can be performed at any temperature. In some cases, a swab used to obtain a sample from the sample collection surface of a feeding device provided herein can be sonicated at about 37° C. In cases where two or more sonication steps are performed, the swab can be maintained at about 37° C. between each sonication step.

In some cases, a sample that was obtained from a sample collection surface of a device provided herein (e.g., a device such as a feeding device having at least one sample collection surface) can be concentrated (e.g., vacuum concentrated) prior to assessing the sample for the presence or absence of one or more misfolded polypeptides (e.g., Prp^{Sc}). For example, an extract obtained from a swab used to obtain a sample from the sample collection surface of a feeding device provided herein can be concentrated (e.g., vacuum concentrated) prior to assessing the sample for the presence or absence of one or more misfolded polypeptides (e.g., Prp^{Sc}). In some cases, a concentration step (e.g., vacuum concentration) can include centrifugation. In some cases, a concentration step (e.g., vacuum concentration) can include evaporation (e.g., solvent evaporation). In some cases, a concentration step (e.g., vacuum concentration) can be performed in the absence of an eluate (e.g., such that the concentration step is not dependent on the chemical composition of an eluate). In some cases, a concentration step (e.g., vacuum concentration) can remove a liquid phase of a sample and can retain a solid phase of the sample. A concentration step (e.g., vacuum concentration) can be performed for any amount of time. For example, an extract obtained from a swab used to obtain a sample from the sample collection surface of a feeding device provided herein can be vacuum concentrated for from about 3 hours to about 5 hours. A concentration step (e.g., vacuum concentration) can be performed at any temperature. For example, an extract obtained from a swab used to obtain a sample from the sample collection surface of a feeding device provided herein can be vacuum concentrated at 45° C. For example, an extract obtained from a swab used to obtain a sample from the sample collection surface of a feeding device provided herein can be vacuum concentrated at about 65° C. In some cases, a concentration step (e.g., vacuum concentration) can concentrate a sample by from about 10 fold to about 100 fold (e.g., from about 10 fold to about 90 fold, from about 10 fold to about 80 fold, from about 10 fold to about 70 fold, from about 10 fold to about 60 fold, from about 10 fold to about 50 fold, from about 10 fold to about 40 fold, from about 10 fold to about 30 fold, from about 10 fold to about 20 fold, from about 20 fold to about 100 fold, from about 30 fold to about 100 fold, from about 40 fold to about 100 fold, from about 50 fold to about 100 fold, from about 60 fold to about 100 fold, from about 70 fold to about 100 fold, from about 80 fold to about 100 fold, from about 90 fold to about 100 fold, from about 20 fold to about 90 fold, from about 30 fold to about 80 fold, from about 40 fold to about 70 fold, from about 50 fold to about 60 fold, from about 20 fold to about 40 fold, from about 30 fold to about 50 fold, from about 40 fold to about 60 fold, from about 50 fold to about 70 fold, from about 60 fold to about 80 fold, or from about 70 fold to about 90 fold).

In some cases, a sample that was obtained from a sample collection surface of a device provided herein (e.g., a device such as a feeding device having at least one sample collection surface) can be digested (e.g., digested with an enzyme such as proteinase K) prior to assessing the sample for the presence or absence of one or more misfolded polypeptides (e.g., Prp^{Sc}).

In some cases, a swab used to obtain a sample from a sample collection surface of a feeding device provided herein (e.g., a device such as a feeding device having at least one sample collection surface) or a sample extracted from a swab used to obtain a sample from a sample collection surface of a feeding device provided herein (e.g., a device such as a feeding device having at least one sample collection surface) can be stored prior to assessing the sample for the presence or absence of one or more misfolded polypeptides (e.g., Prp^{Sc}). For example, a swab can be stored in moist conditions (e.g., in a buffer solution such as a phosphate-buffered saline (PBS) solution) prior to assessing the sample for the presence or absence of one or more misfolded polypeptides (e.g., Prp^{Sc}). For example, a swab can be stored at temperatures of from about −80° C. to about 4° C. (e.g., at about −80° C., at about −20° C., and at about 4° C.) prior to assessing the sample for the presence or absence of one or more misfolded polypeptides (e.g., Prp^{Sc}). In some cases, a swab can be stored at temperatures of from about −80° C. to about 4° C. for any amount of time prior to assessing the sample for the presence or absence of one or more misfolded polypeptides (e.g., Prp^{Sc}).

Any appropriate method can be used to detect the presence or absence of one or more misfolded polypeptides (e.g., Prp^{Sc}) in a sample from one or more mammals (e.g., one or more cervids) that was obtained from a sample collection surface of a device provided herein (e.g., a device such as a feeding device having at least one sample collection surface). For example, RT-QuIC, enzyme-linked immunosorbent assay (ELISA), immunohistochemistry (IHC), protein misfolding cyclic amplification (PMCA), and/or western blot tests can be used to detect of the presence or absence of one or more misfolded polypeptides (e.g., Prp^{Sc}) in a sample from one or more mammals (e.g., one or more cervids) that was obtained from a sample collection surface of a feeding device provided herein.

The methods and materials provided herein can be used to detect the presence or absence of any misfolded polypeptide in a sample from one or more mammals (e.g., one or more cervids) that was obtained from a sample collection surface of a device provided herein (e.g., a device such as a feeding device having at least one sample collection surface). In some cases, a misfolded polypeptide can be associated with a disease (e.g., a prion disease). Examples of polypeptides that can be misfolded, and where the misfolded polypeptide can be detected using the methods and materials described herein include, without limitation, Prp^{Sc}, misfolded polypeptides associated with transmissible spongiform encephalopathies (PrP^{TSE}), misfolded polypeptides associated with chronic wasting disease (Prp^{CWD}), misfolded polypeptides associated with bovine spongiform encephalopathy (PrP^{BSE}), misfolded polypeptides associated with Creutzfeldt-Jakob Disease (PrP^{CJD}), misfolded polypeptides associated with feline spongiform encephalopathy (PrP^{FSE}), misfolded polypeptides associated with transmissible mink encephalopathy (PrP^{TME}), misfolded polypeptides associated with camel spongiform encephalopathy (PrP^{CSE}), misfolded tau polypeptides, misfolded alpha-synuclein polypeptides, and misfolded amyloid beta polypeptides.

The methods described herein (e.g., the methods for detecting the presence or absence of a misfolded polypeptide) can be used to detect the presence or absence of a misfolded polypeptide associated with any prion disease. As used herein, a prion disease is any disease associated with misfolding and, optionally, aggregation of one or more of the misfolded polypeptides (e.g., Prp^{Sc}). Examples of prion diseases associated polypeptides that can be misfolded, and where the misfolded polypeptide can be detected as described herein include, without limitation, CWD, TME, BSE, scrapie, feline spongiform encephalopathy, ungulate spongiform encephalopathy, camilid spongiform encephalopathy, Creutzfeldt-Jakob disease (CJD), Gerstmann-Straussler-Scheinker disease (GSS), fatal familial insomnia (FFI), Alzheimer's disease, and Parkinson's disease.

This document also provides methods and materials for monitoring a population of mammals (e.g., a herd of cervids) for the presence or absence of one or more prion diseases as described herein (e.g., based, at least in part, on the presence or absence of one or more misfolded polypeptides (e.g., Prp^{Sc}) in a sample from one or more mammals that was obtained from a device provided herein). In some cases where a population of mammals (e.g., a herd of cervids) is identified as having one or more prion diseases based, at least in part, on the presence of one or more misfolded polypeptides (e.g., Prp^{Sc}) in a sample that was obtained from a device provided herein (e.g., a device such as a feeding device having at least one sample collection surface), a population of mammals (e.g., a herd of a cervids) that feeds from the feeding device can be monitored for the presence or absence of one or more prion diseases more frequently. For example, a population of mammals (e.g., a herd of cervids) that has been identified as having one or more prion diseases as described herein (e.g., based, at least in part, on the presence or absence of one or more misfolded polypeptides (e.g., Prp^{Sc}) in a sample from one or more mammals that was obtained from a device provided herein) can be assessed for the presence or absence of one or more prion diseases from about every day to about every month (e.g., daily, weekly, or monthly).

In some cases where a population of mammals (e.g., a herd of cervids) is identified as having one or more prion diseases based, at least in part, on the presence of one or more misfolded polypeptides (e.g., Prp^{Sc}) in a sample that was obtained from a device provided herein (e.g., a device such as a feeding device having at least one sample collection surface), one or more additional populations of mammals (e.g., one or more additional herds of cervids) living nearby can be assessed for the presence or absence of one or more prion diseases to monitor for any spread of the one or more prion diseases. For example, one or more populations of mammals (e.g., one or more herds of cervids) that live from about 1 mile to about 60 miles (e.g., from about 1 mile to about 50 miles, from about 1 mile to about 40 miles, from about 1 mile to about 30 miles, from about 1 mile to about 20 miles, from about 1 mile to about 10 miles, from about 10 miles to about 60 miles, from about 20 miles to about 60 miles, from about 30 miles to about 60 miles, from about 40 miles to about 60 miles, from about 50 miles to about 60 miles, from about 10 miles to about 50 miles, from about 20 miles to about 40 miles, from about 10 miles to about 20 miles, from about 20 miles to about 30 miles, from about 30 miles to about 40 miles, or from about 40 miles to about 50 miles) from a population of mammals (e.g., a herd of cervids) that has been identified as having one or more prion diseases as described herein (e.g., based, at least in part, on the presence or absence of one or more misfolded polypeptides (e.g., Prp^{Sc}) in a sample from one or more mammals that was obtained from a device provided herein) can be assessed for the presence or absence of one or more prion diseases. For example, one or more populations of mammals (e.g., one or more herds of cervids) that live adjacent to (e.g., adjacent to and separated by a barrier such as a fence) a population of mammals (e.g., a herd of cervids) that has been identified as having one or more prion diseases as described herein (e.g., based, at least in part, on the presence or absence of one or more misfolded polypeptides (e.g., PrPSc) in a sample from one or more mammals that was obtained from a device provided herein) can be assessed for the presence or absence of one or more prion diseases

The invention will be further described in the following examples, which do not limit the scope of the invention described in the claims.

### EXAMPLES

### Example 1: Quantitative Measurements of Chronic Wasting Disease Prions Recovered from Swab Samples and Environmentally Relevant Surfaces

This example describes a rapid method for obtaining prions by swabbing different types of surfaces, extracting prions from the swabs, and quantifying the swab-recovered CWD prions.

### Materials and Methods

### Prion Sources and Tissue Preparation

Brain tissues were collected from hamsters infected with the hyper (HY) strain of transmissible mink encephalopathy (TME) and from three elk infected with CWD designated as isolate CWD t1821, CWD JB R296, CWD JB B188, and CWD 307 CL. Brain tissue was homogenized to either 10 or 20% (w/v) in Dulbecco's phosphate-buffered saline (DPBS) without Ca²⁺ or Mg²⁺ (Mediatech, Herndon, VA) or PBS using strain-dedicated Tenbroeck tissue grinders (Kontes, Vineland, NJ) or with Beadblaster 24 Microtube Homogenizer (D2400, Benchmark Scientific, Inc., Sayreville, NJ, US). Samples were stored at −80° C. until use.

### Swab Contamination

Cotton-tipped swabs (3M™ Quick Swab, 3M, Saint Paul, MN, US) and foam-tipped swabs (Fisherbrand™ PurSwab Foam Swabs, Cat. No: 14-960-3E, Thermo Fisher Scientific, Waltham, MA, US) were used. To contaminate the swabs with prions, 500 (HY TME, CWD JB B188, and CWD JB R296) or 1000 (CWD t1821) μg brain equivalents (BE) of brain homogenate (BH) was applied to the surface of swabs by pipet. Contaminated swabs in triplicate were incubated at room temperature to dry for 0 hours (undried control), 0.25 hours, 0.5 hours, 1 hour, 6 hours, 12 hours, or 24 hours. After drying, swabs were immediately stored in 1.7 mL microcentrifuge tubes with 300 μL (for foam swabs) or 500 μL (for cotton swabs) of DPBS to completely cover the swab tips. Handles of swabs were cut to fit in the tube with the cap closed.

### Surface Contamination

Ten-fold serial dilutions of brain homogenates ranging from 500 to 0.5 (HY TME, CWD JB B188, and CWD JB R296) or 1000 to 1 (CWD t1821) μg BE were applied to glass slides (Fisherbrand Superfrost Plus Microscope slides, Catalog No.: 12-550-15), stainless steel (316L grade, Millard Metal Services, La Vista, NE, US), or oak wood coupons (Lowe's, Omaha, NE, US) by pipet. Contaminated surfaces were dried at room temperature for 24 hours then sampled with foam-tipped swabs. A swab was wetted with ultrapure water then applied to the surface 10 times with spinning to maximize the exposure to the swab surface. To prepare positive controls, surfaces contaminated with the highest levels of prions (500 μg BE for CWD JB B188 and CWD JB R296; 1000 μg BE for CWD t1821) were sampled with dry swabs immediately following contamination, in a manner to absorb all liquid. Surfaces without prion contamination (negative controls) were sampled as described for the prion-contaminated surfaces. To prepare prion-contaminated surface for RT-QuIC detection, 50 μL from each dilution of CWD 307 CL (log −2 to −6) were applied to stainless steel and dried at 22° C. for 24 hours followed by swabbing with foam-tipped swabs as described above. Surfaces were prepared in triplicate for each sample including positive and negative controls. One swab was used for each area as subsequent swabs could not recover detectable prions according to previous experiments (Table 1). Swab tips cut to fit the tubes were placed in 1.7 mL microcentrifuge tubes with 300 μL of DPBS in each to cover the foam tips and extracted immediately.

TABLE 1
No detectable CWD^{a} Prp^{Sc} from the 2^{nd} swab^{b}
of contaminated surfaces by 96-well immunoblot assay.

| | Undried | 12-hr dried |
|---|---|---|
| Glass | −0.6% ± 0.3%^{c} | −1.5% ± 0.2% |
| Stainless Steel | −1.1% ± 0.5% | −1.1% ± 0.2% |
| Wood | −1.4% ± 0.3% | −1.1% ± 0.3% |

^{a}CWDt1821 was tested.
^{b}For undried surfaces, the 2^{nd} swab was dry; for 12-hr dried surfaces, the 2^{nd} swab was wetted with ultrapure water.
^{c}Ratio of detected CWD Prp^{Sc} to the abundance of CWD Prp^{Sc} applied to contaminated surfaces in percentage, results were expressed as Mean ± SEM, n = 4.

### Swab Extraction

Swabs in microcentrifuge tubes were either incubated with shaking or sonication for prion extraction. Four replicate swabs were prepared for the swab drying experiments. With shaking extraction, undried or dried swabs were incubated in 300 μL (for foam swabs) or 500 μL (for cotton swabs) of DPBS, and were agitated on a micro tube mixer (Tomy MT-360, speed 5) at room temperature for 30 minutes for the 1st extraction. This was followed with 2 serial extractions incubating the swabs in a different microcentrifuge tube with 200 μL (for foam swabs) or 300 μL (for cotton swabs) of DPBS for 30 minutes for each extraction. With sonication extraction, swabs stored in microcentrifuge tubes were placed in a QSonica sonicator (model Q700) with amplitude set to level 17, generating an average output of around 170 W during sonication treatment. Depending on desired length of total treatment time for each extraction (5 seconds, 15 seconds, 30 seconds, or 60 seconds), sonication were performed at 37° C. and consisted of different numbers (1, 3, 6, or 12) of treatment cycles (5 seconds of sonication followed by 5 seconds of incubation). For each swab, three serial extractions with the same length of sonication time were applied. The buffer usage for sonication extraction was also the same as for shaking. Extracts (˜200 μL each for foam swabs, ˜300 μL each for cotton swabs) were stored at −80° C. until ready for concentration. Swabs used for surface sampling were extracted twice (one time for swabs of negative surface controls) using 15 seconds of sonication for each extraction (3 treatment cycles). Extracts were combined (˜400 μL) for each swab and stored at −80° C.

Swabs used for surface sampling were extracted twice (one time for swabs of negative surface controls for immunodetection) using 15 s of sonication for each extraction (3 treatment cycles). Extracts were either combined (˜400 μL for swabs for immunodetection) or stored separately (˜200 μL for swabs for RT-QuIC). All extracts were stored at −80° C.

### Extracts Concentration

All extracts were vacuum concentrated with Savant Speed-Vac concentrator equipped with Savant refrigerated vapor trap (RVT4104). Samples were evaporated at high rate (65° C. in the chamber) for 3 hours, 4 hours, or 5 hours for 200 μL, 300 μL, or 400 μL of extracts, respectively. Extracts for RT-QuIC detection were vacuum concentrated with SpeedVac (SPD 1030, Thermo Fisher Scientific, Waltham, MA, US). Samples were evaporated at 45° C. for 2 hours with 10 Torr vacuum and rehydrated with 50 μL of ultrapure water. Concentrated extracts were rehydrated with 10 μL, 15 μL, or 20 μL of ultrapure water correspondingly and stored at −80° C. before analysis.

### Detection and Quantification

Samples were digested with 23.25 μg/mL (HY TME, CWD JB B188, and CWD JB R296) or 46.5 μg/mL (CWD t1821) proteinase K (PK) (Roche Diagnostics Corporation, Indianapolis, IN) at 37° C. for 30 minutes with constant agitation. 96-well immunoblot assay was performed with primary monoclonal antibody 3F4 (Sigma-Aldrich, St. Louis, MO, US; 0.1 μg/mL, 37° C. for 1 hour) for HY TME or 8H4 (Sigma-Aldrich, St. Louis, MO, US; 0.17 μg/mL, 37° C. for 1 hour) for CWD and secondary antibody (horseradish-peroxidase conjugated anti-mouse IgG, Invitrogen, Carlsbad, CA, US; 0.01 μg/mL, 37° C. for 30 minutes). Well-plate membranes were developed with Supersignal West Femto maximum sensitivity substrate, according to the manufacturer's instructions (Pierce, Rockford, IL, US), imaged on a 4000R imaging station (Kodak, Rochester, NY), and analyzed using Kodak (New Haven, CT) molecular imaging software (V.5.0.1.27) which output the net intensity of each well. For each plate, 2-fold dilutions of the control were prepared to generate a linear regression of Prp^{Sc} abundance used for the quantification of sample intensities (standard curve). An aliquot of recovered Prp^{Sc} from swabs or surfaces (e.g., 1/10 in order to generate a signal within the linear range) was loaded to the wells and calculated for Prp^{Sc} amount according to the standard curve. The total amount of recovered Prp^{Sc} were then adjusted for aliquoting. The unit of Prp^{Sc} used in this study was μg brain equivalent (BE) referring to Prp^{Sc} content in corresponding μg of wet weight of the brain tissue. The recovery was determined as the ratio of total amount of recovered Prp^{Sc} to the amount of Prp^{Sc} originally loaded. Statistical analysis was performed with unpaired parametric Welch's t-test using GraphPad Prism 8.

### Results

Surface Drying Decreased Prion Recovery from Swabs Extracted Under Gentle Shaking

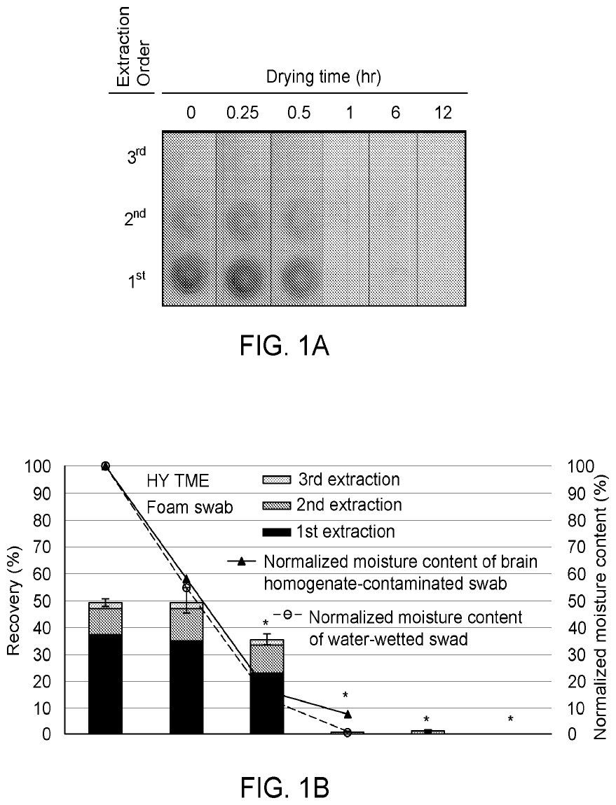

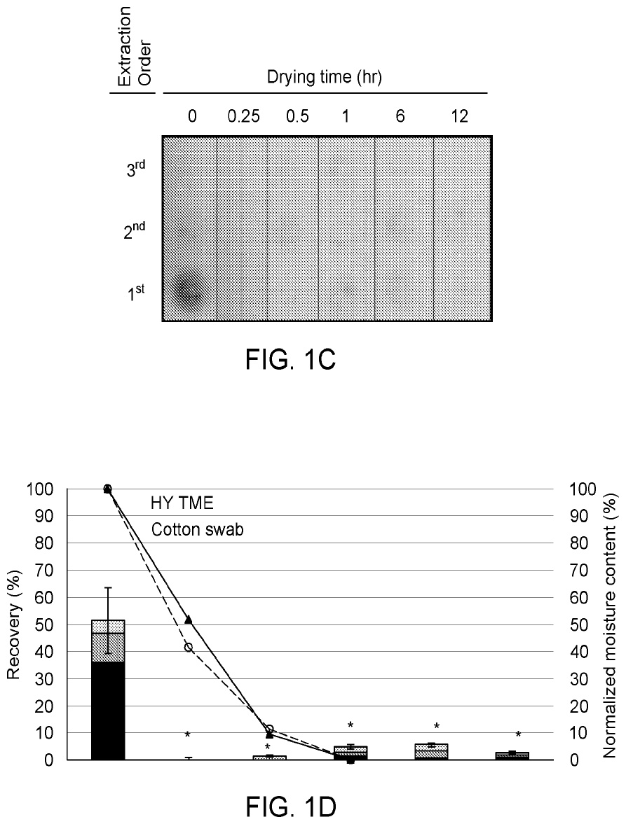

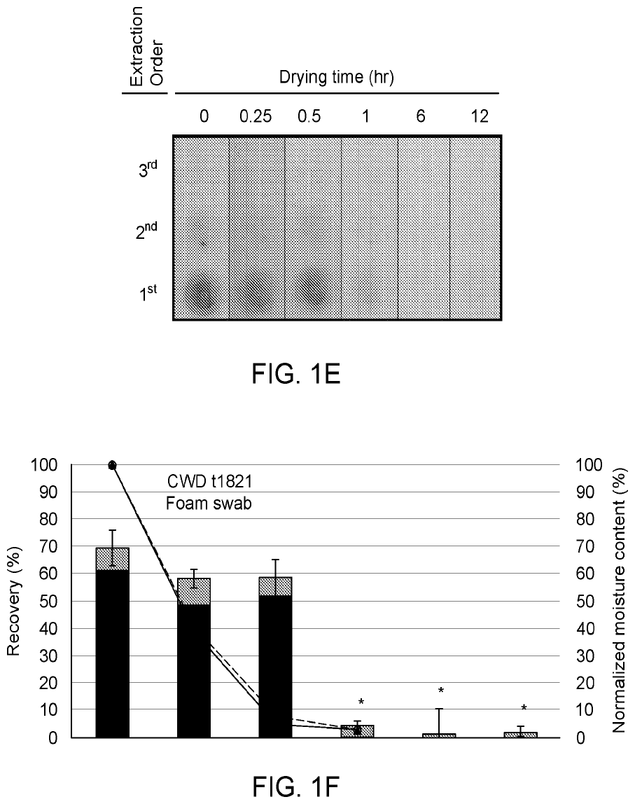

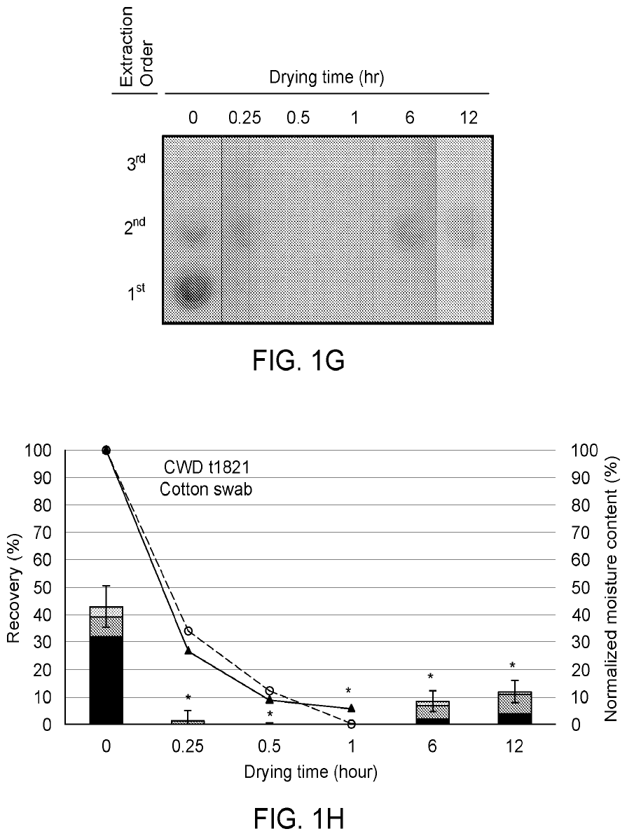

The detection and quantification of HY Prp^{Sc} (FIGS. 1A, 1B, 1C, and 1D) and CWD t1821 Prp^{Sc} (FIGS. 1E, 1F, 1G, and 1H) from each extraction and/or 3 sequential extractions of the swabs dried for different times are presented. The recovery of HY Prp^{Sc} from the foam swab without drying (0 hours) was 49%±1% (mean±standard error of mean) (FIG. 1B). After 15 minutes of drying (0.25 hours), HY Prp^{Sc} recovery from the foam swab maintained similarly (p>0.05) at 50%±4% and significantly decreased (p<0.05) to 36%±2% after 30 minutes of drying (0.5 hours) (FIG. 1B). After 1 hour of drying, the extracted HY Prp^{Sc} was near the limit of Prp^{Sc} detection (FIG. 1A) and the recovery reduced significantly (p<0.05) to approximately 1% (FIG. 1B). Application of HY to cotton swabs (FIGS. 1C and 1D) resulted in 51%±12% recovery of Prp^{Sc} without drying (0 hours) that after 15 minutes of drying (0.25 hours) was significantly decreased (p<0.05) to less than 6%. Similar results were observed for CWD. From foam swabs, total recoveries of CWD Prp^{Sc} after 0 hours, 0.25 hours, and 0.5 hours of drying were 68%±7%, 58%±3%, and 59%±7%, respectively (FIGS. 1E and 1F). After 1 hour of drying, the extracted CWD Prp^{Sc} significantly decreased (p<0.05) to less than 5% (FIGS. 1E and 1F). From cotton swabs, 43%±3% of CWD Prp^{Sc} was recovered without drying (0 hours) and the total CWD recoveries thereafter significantly reduced (p<0.05) to 12% or lower (FIGS. 1G and 1H).

To investigate if the difference between foam swabs and cotton swabs is related to drying dynamics, the moisture content of both types of swab containing either HY or CWD Prp^{Sc} or the same volume of ultra-purified water were determined. The overall trends of swab moisture content are similar among all conditions, with a moisture content reduction to approximately 10% after 30 minutes of surface drying and a further reduction through 1 hour of drying (FIG. 1), suggesting that differences in moisture content between the cotton and foam swabs did not affect prion recovery.

Enhancement of HY Recovery from Dried Foam Swabs Using Sonication

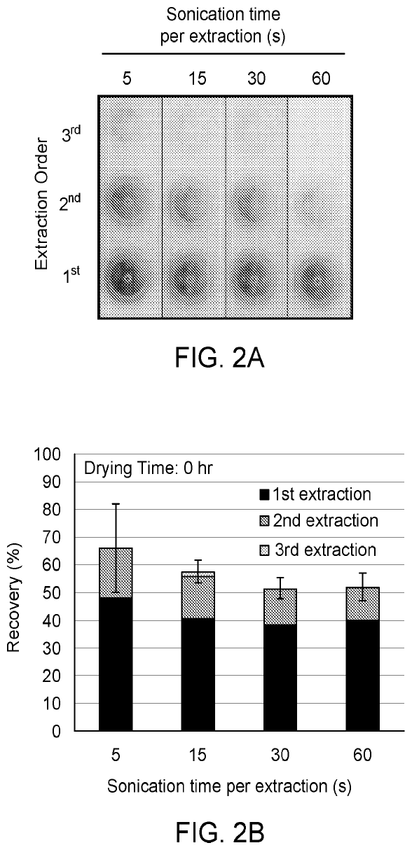

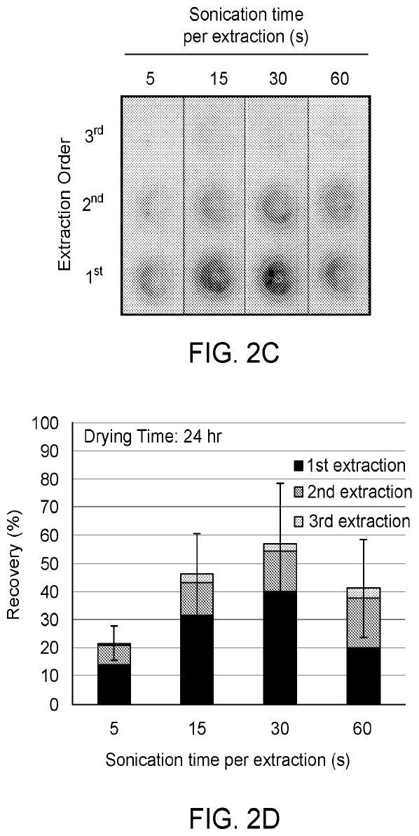

To investigate whether a more energetic mechanical force can improve the extraction efficiency for surface-dried Prp^{Sc}, sonication was used to replace the gentle shaking. Sonication of foam swabs for 5 seconds, 15 seconds, 30 seconds, and 60 seconds immediately after application of HY (FIGS. 2A and 2B), resulted in total HY Prp^{Sc} recoveries of 66%±16%, 58%±4%, 51%±4%, and 52%±5%, respectively. Recoveries of HY Prp^{Sc} were similar (p>0.05) between the tested sonication durations and that from undried foam swabs extracted with gentle shaking (FIGS. 1A and 1B). After 24 hours of drying on foam swabs, total HY Prp^{Sc} recoveries were 22%±6%, 46%±14%, 57%±21%, and 41%±18% for the tested sonication durations from short (5 seconds) to long (60 seconds). The recovery of Prp^{Sc} from the sonication-treated dried foam swabs was generally significantly (p<0.05) greater than from dried swabs (≥1 hour) extracted with gentle shaking (FIGS. 1A and 1B). Overall, sonication increased the recovery of Prp^{Sc} from dried foam swabs.

Recovery of Surface-Dried CWD from Foam Swabs by Sonication Extraction Varied Among Isolates

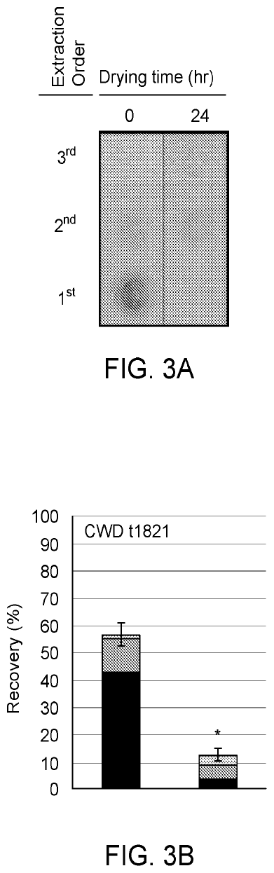

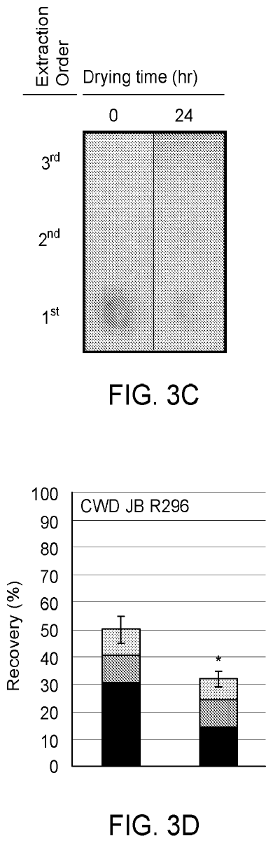

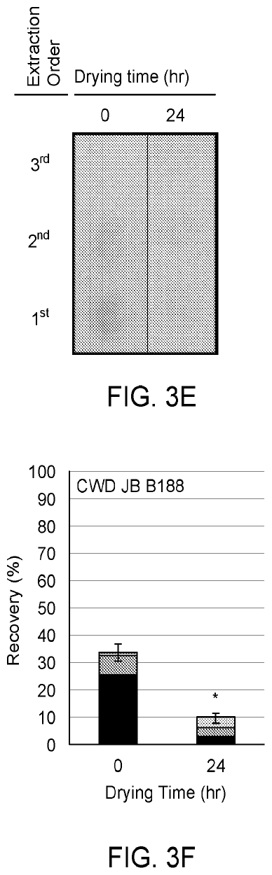

To examine the efficiency of sonication extraction for surface-dried CWD from foam swabs, CWD contaminated swabs were either extracted immediately (0 hours) or after 24 hours of drying (24 hours) with 15 seconds of sonication. After three sequential extractions, total CWD Prp^{Sc} recoveries from undried foam swabs were 57%±4% for CWD t1821, 50%±5% for CWD JB R296, and 34%±3% for CWD JB B188 (FIG. 3). After 24-hour drying, total CWD Prp^{Sc} recoveries decreased significantly (p<0.05) to 13%±2%, 32%±3%, and 10%±2%, respectively. Total recovery of Prp^{Sc} from the undried foam swabs contaminated with either CWD t1821 or CWD JB R296 were significantly (p<0.05) higher compared to foam swabs contaminated with CWD JB B188, whereas from 24-hour dried foam swab, total recovery of CWD JB R296 was higher (p<0.05) than the other samples. Recovery of CWD Prp^{Sc} from dried swabs was enhanced with sonication extraction. From undried foam swabs, total CWD t1821 recovery was similar (p>0.05) between with gentle shaking and with sonication (FIGS. 1E and 1F and FIGS. 3A and 3B). Sonication extraction from dried foam swabs resulted in greater (p<0.05) CWD Prp^{Sc} recovery compared to shaking extraction (FIGS. 1E and 1F and FIGS. 3A and 3B).

Prion Recovery from Glass and Stainless Steel are Higher than from Wood

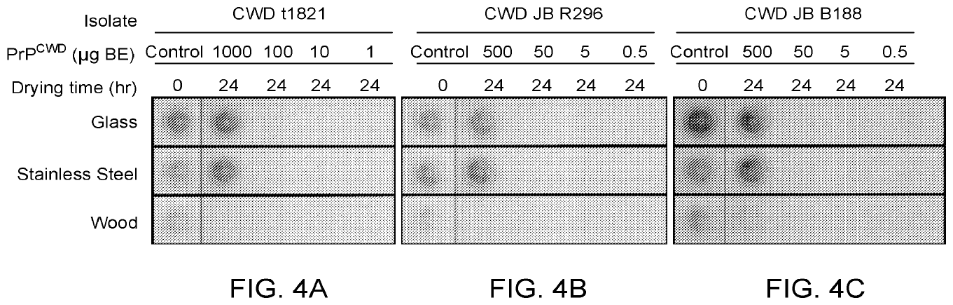

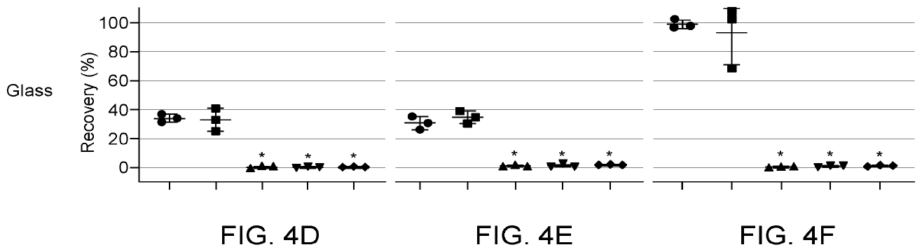

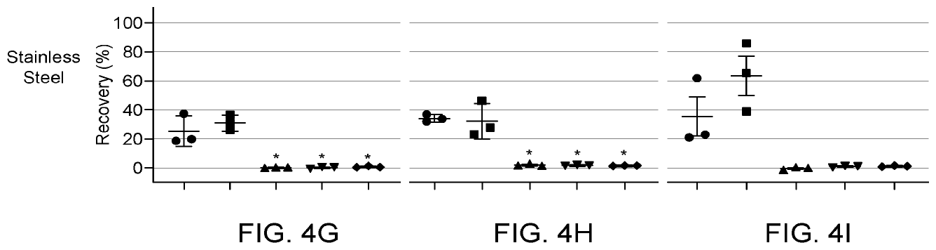

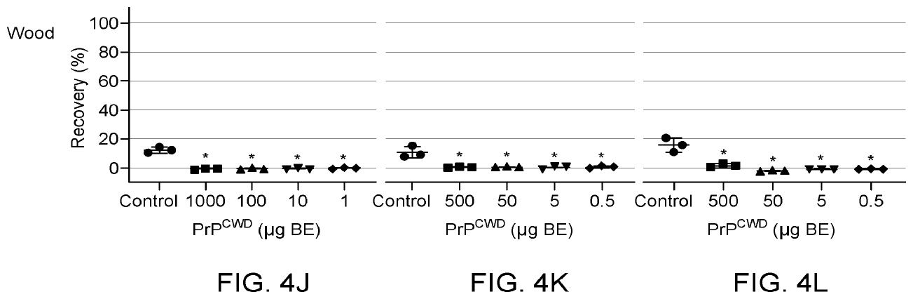

Prions from three environmentally relevant surfaces contaminated with three CWD isolates were sampled using foam swabs. Prp^{Sc} was extracted from the swab using short sonication extractions (15 seconds) (FIG. 4). With immediate swabbing after surface contamination with the highest level of CWD prions (1000 μg brain equivalents (BE) for CWD t1821, 500 μg BE for CWD JB R296 and CWD JB B188), and immediate swab extraction (control), recoveries of CWD Prp^{Sc} ranged from 25% to 99% for glass and stainless steel surfaces, with the majority around 30% (Table 2 and FIGS. 4A-4I). In contrast, contamination of wood with CWD resulted in significantly lower (p<0.05, except for FIG. 4G vs. 4J and 4I vs. 4L) Prp^{Sc} recoveries for all CWD isolates tested (11%˜16%) from undried surface (control) (Table 2 and FIGS. 4A-4C, and 4J-4L). At the highest contamination levels, all CWD isolates had similar (p>0.05) recovery from glass and stainless steel after 24 hours of drying compared to their controls (Table 2 and FIGS. 4A-4I). However, extracted CWD Prp^{Sc}, at the highest contamination levels, from wood after 24 hours of drying were significantly reduced (p<0.05) compared to their controls (Table 2 and FIGS. 4A-4C and 4J-4L). At all other lower contamination levels, CWD Prp^{Sc} was not detected regardless of the surface type. Swab extracts from surfaces without prion contamination (negative controls) generated undetectable CWD Prp^{Sc} (Table 3) for all surface types.

TABLE 2
Recovery of PrP^{Sc} from three CWD isolates from glass, stainless
steel, and wood without (0 hours) and with drying (24 hours).

| Surface | CWD t1821 (%) | | CWD JB R296 (%) | | CWD JB B188 (%) | |
|---|---|---|---|---|---|---|
| Type | 0 hours^{a} | 24 hours^{b} | 0 hours^{a} | 24 hours^{b} | 0 hours^{a} | 24 hours^{b} |
| Glass | 34 ± 1^{c} | 33 ± 5 | 31 ± 3 | 35 ± 3 | 99 ± 2 | 93 ± 13 |
| Stainless | 25 ± 6 | 31 ± 3 | 34 ± 2 | 32 ± 7 | 36 ± 14 | 64 ± 14 |
| Steel | | | | | | |
| Wood | 12 ± 1 | −1 ± 0 | 11 ± 2 | 1 ± 0 | 16 ± 3 | 2 ± 2 |

^{a}Surfaces were contaminated with 1000 μg BE, 500 μg BE, and 500 μg BE for CWD t1821, CWD JB R296, and CWD JB B188 without drying, designated as “control” in FIG. 4.
^{b}Surfaces were contaminated with the highest prion loads (equivalent to the “control”) with 24 hours of drying.
^{c}Total CWD recoveries expressed as mean ± standard error of mean (SEM), n = 3

TABLE 3
No detectable CWD Prp^{Sc} from uncontaminated
surfaces by 96-well immunoblot assay.

| | Glass | Stainless Steel | Wood |
|---|---|---|---|
| Area 1 | −0.3%^{a} | −1.8% | 1.1% |
| Area 2 | 2.7% | 0.1% | −0.4% |
| Area 3 | −0.5% | −0.8% | −0.7% |

^{a}Ratio of detected CWD Prp^{Sc} to the abundance of CWD Prp^{Sc} applied to contaminated surfaces in percentage.

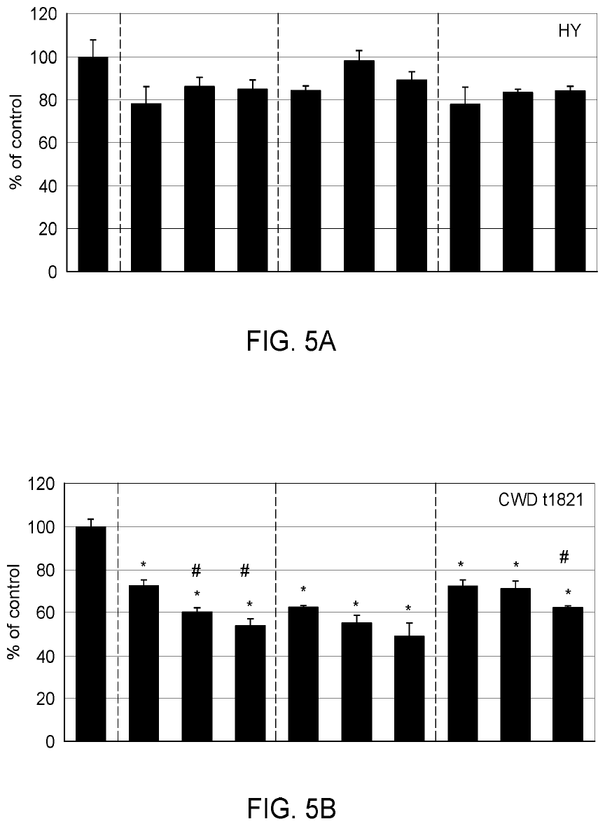

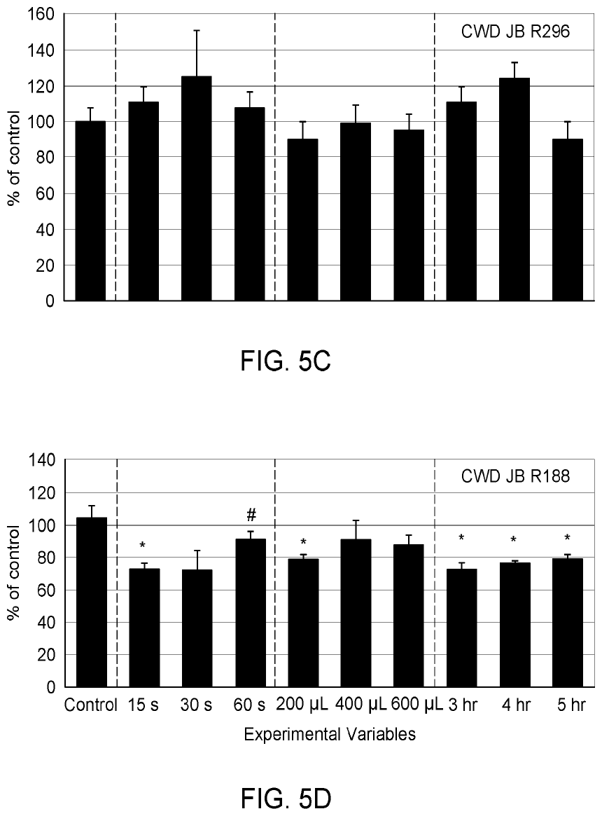

In the swab-free experimental system, 10 to 50% of Prp^{Sc} of two CWD isolates was not recovered after undergoing the aforementioned processes (FIGS. 5B and 5D).

However, this contribution might be limited and/or vary with prion strains/isolates as HY and one CWD isolate were not significantly reduced (p>0.05) (FIGS. 5A and 5C).

Together these results demonstrate that misfolded polypeptides (e.g., CWD prions) can be recovered from stainless steel, glass, and wood using swabs. Methods and materials for recovering misfolded polypeptides can be used for prion detection and, optionally, monitoring in a natural environment.

### Example 2: RT-QuIC Detection Sensitivity for CWD Swabbed from Stainless Steel Surfaces

### Materials and Methods

### RT-QuIC Reaction

RT-QuIC was performed and analyzed as described elsewhere (Schwabenlander et al., J. Wildl. Dis., 58(1):50-62 (2022); and Wilham et al., PloS Pathog., 6(12):e1001217 (2010)). Briefly, control serial dilutions and swab extracts from Example 1 were diluted 10-fold in 0.1% SDS/1×PBS/1× N2. 2 μL of control and swab extract diluent were added to 98 μL of RT-QuIC reaction buffer. The reaction buffer was made to the following concentrations: 1×PBS, 170 mM NaCl, 1 mM EDTA, 10 UM thioflavin T (ThT), and 0.1 mg/mL recombinant hamster prion protein (recHaPrP). The reactions were read for ThT fluorescence which emits light with maximum excitation/emission at approximately 450/480 every 45 minutes for 48 hours. Shaking was performed at 700 rpm double orbital for 1 minute and rest for 1 minute. A maxpoint ratio (MPR) was calculated by taking the maximum fluorescence of each well and dividing it by the initial fluorescence (i.e., the background fluorescence) as described elsewhere (Vendramelli et al., J. Clin. Microbiol., 56:e00423-18 (2018)). A rate of amyloid formation (RAF) was also calculated per well as the reciprocal of the time necessary for fluorescence to reach twice the background fluorescence.

### Results

RT-QuIC Detection of CWD) Prion from Swabs and Stainless Steel Surface

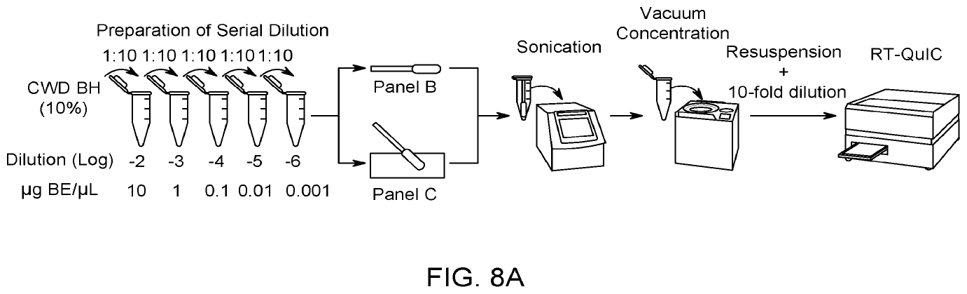

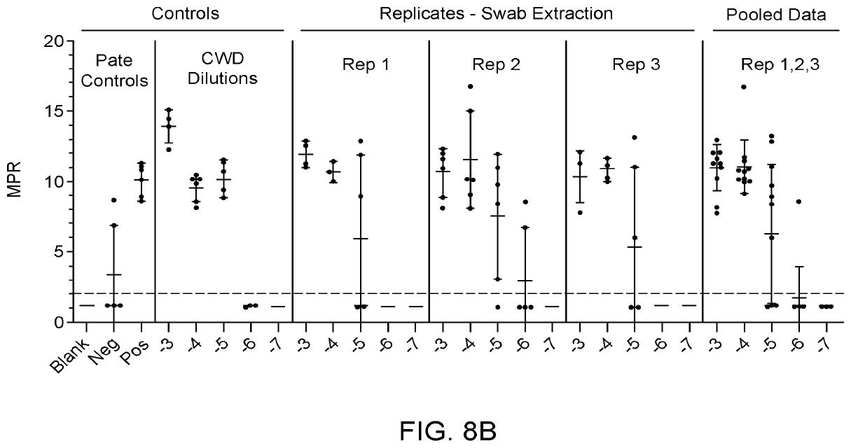

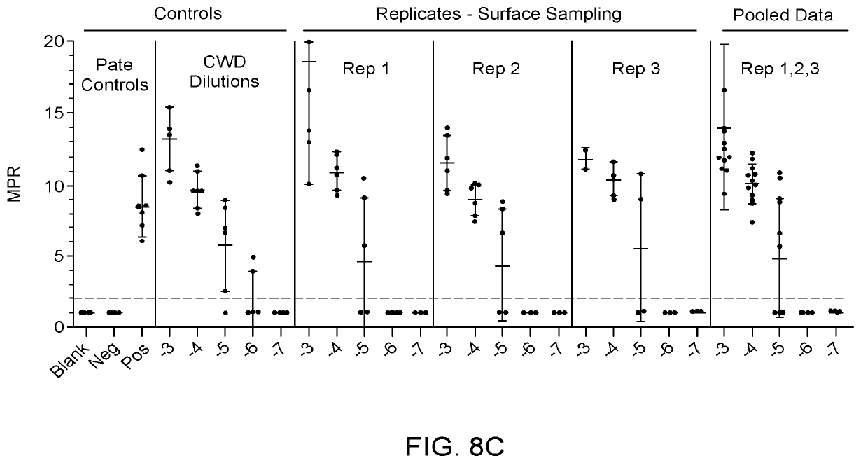

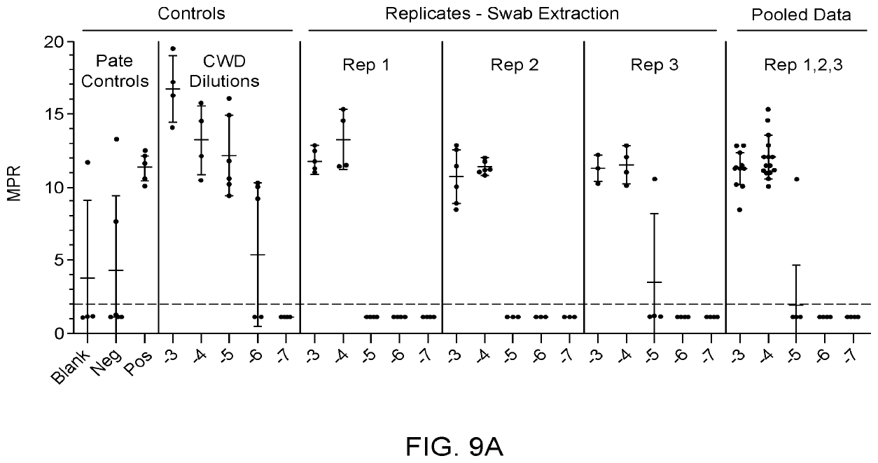

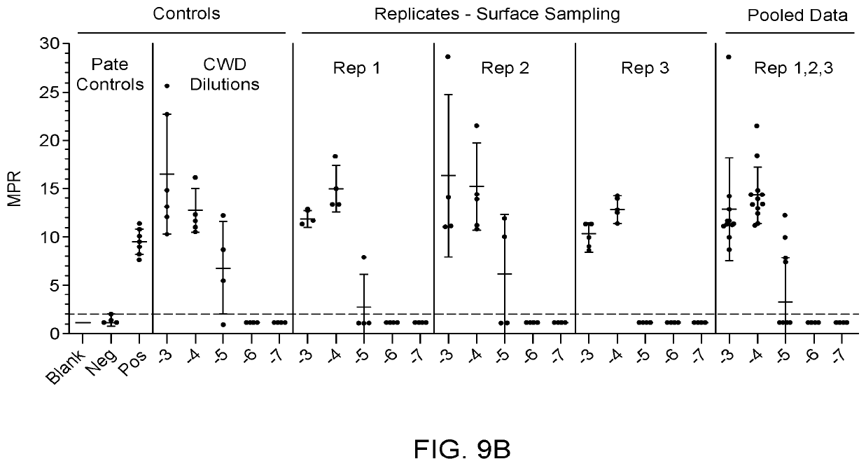

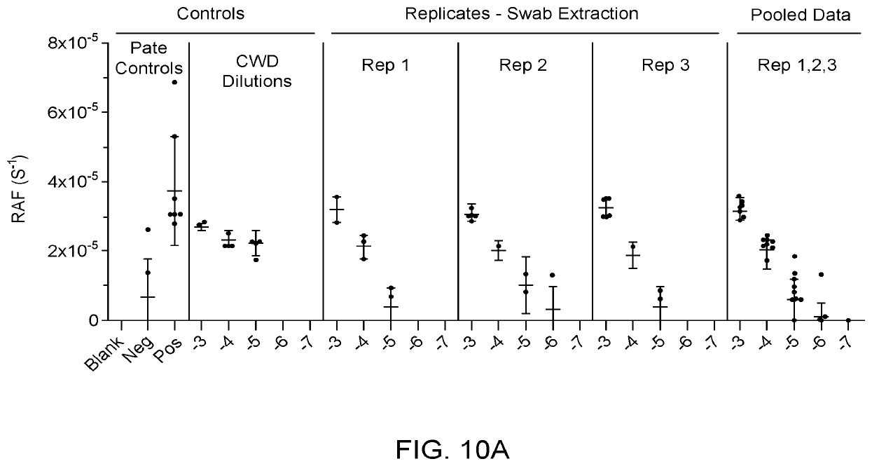

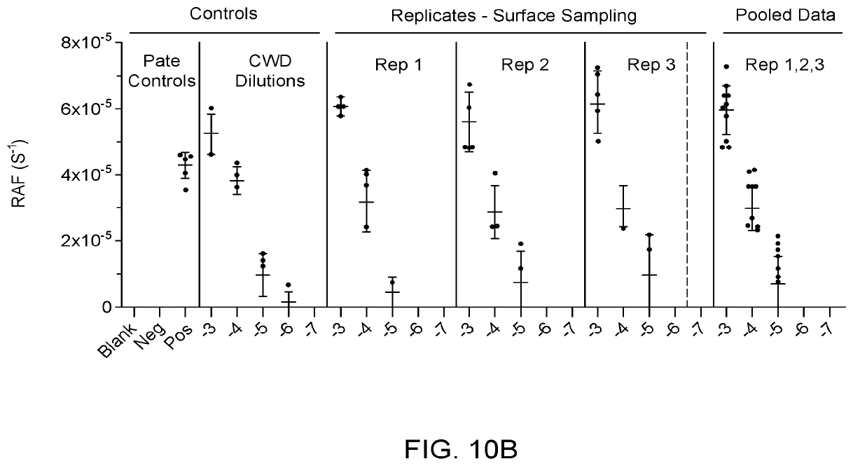

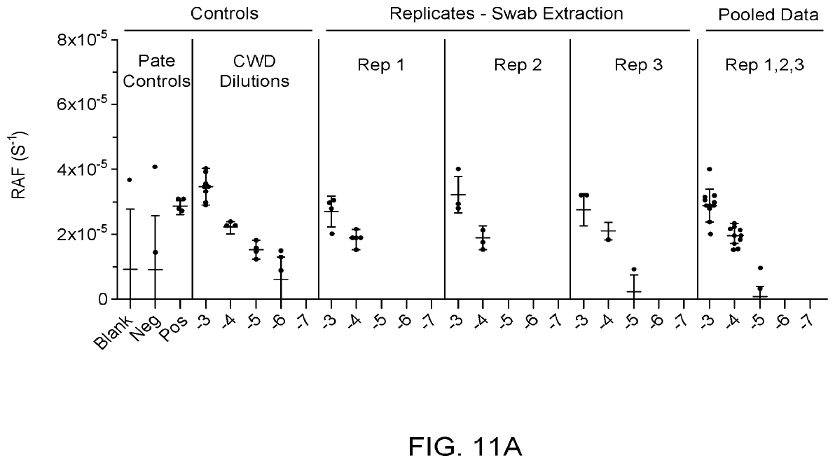

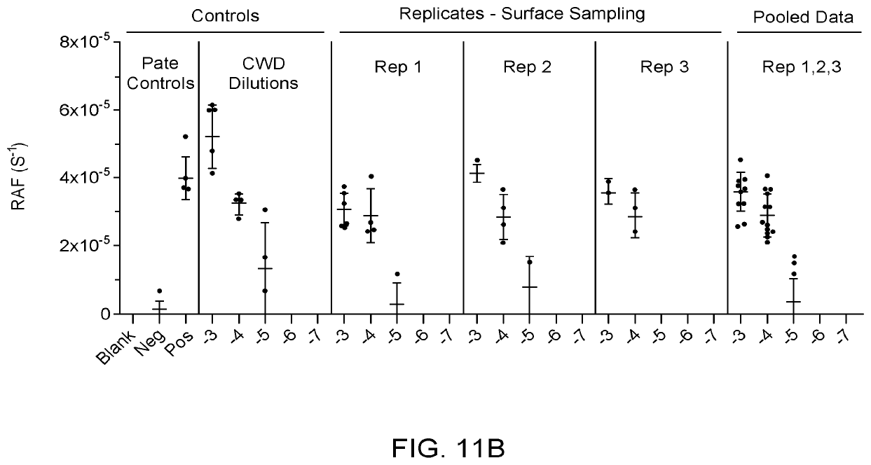

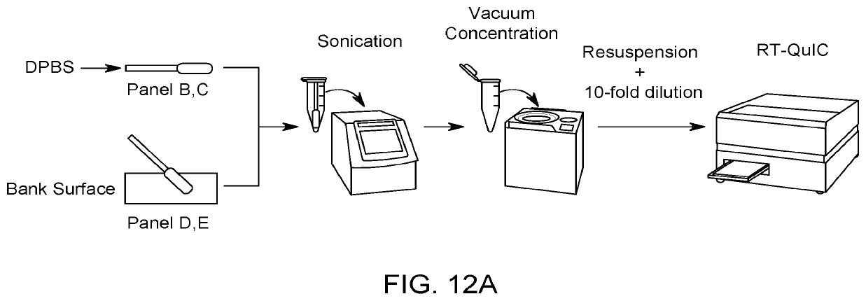

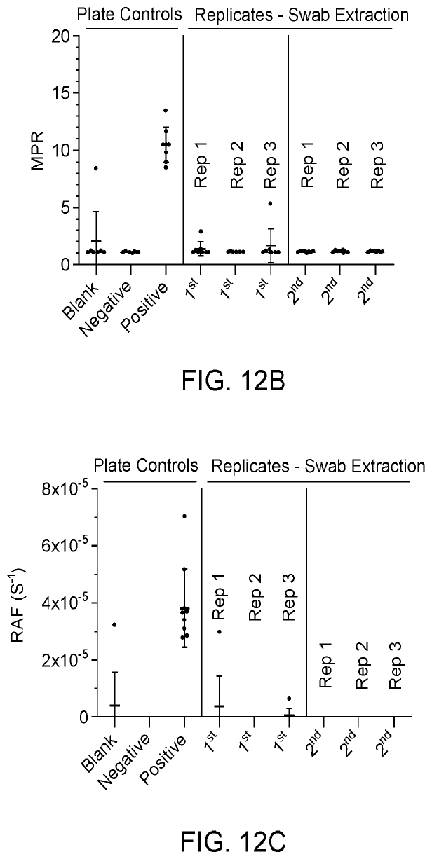

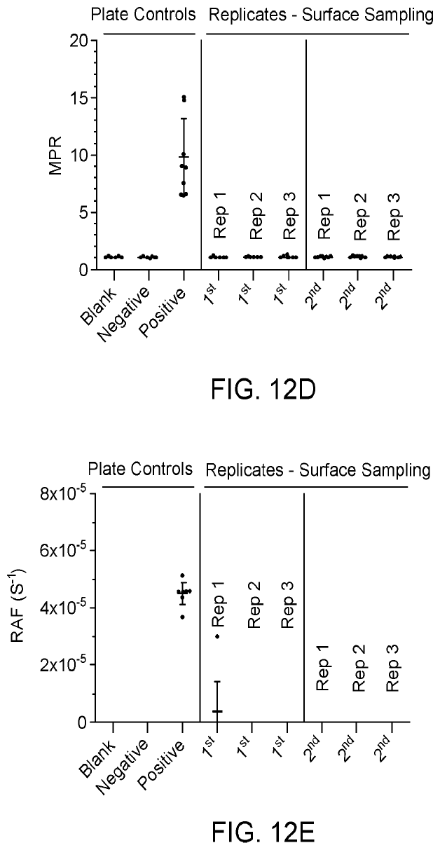

To apply the optimized prion swab extraction method to the detection of prions swabbed from surfaces the following experimental method was employed. First, serial 10-fold dilutions of CWD-infected brain (isolate 307 CL) were prepared ranging from 10-2 (10 μg brain equivalents/ml) to 106 (0.001 μg brain equivalents/ml) (FIG. 8A). These dilutions were directly analyzed by RT-QuIC resulting in a detection limit of 10-5 (FIGS. 8B and 8C controls). Next the same serial 10-fold dilutions of CWD-infected brain were applied to foam-tipped swabs, extracted using the optimized protocol and analysis of the extracts using RT-QuIC resulted in detection limit of 10-5 in all three experimental replicates (FIGS. 8A and 8B). Finally, the same serial 10-fold dilutions of CWD-infected brain were applied to stainless steel surface, dried for 24 hours at 22° C., then the surfaces were swabbed, the swabs were extracted using the optimized protocol and analysis of the extracts using RT-QuIC resulted in detection limit of 10-5 in all three experimental replicates (FIGS. 8A and 8C). The RT-QuIC CWD detection limit was similar between the direct analysis of the serial 10-fold dilutions of CWD-infected brain, the analysis of CWD directly applied to swabs (FIG. 8B pooled) and CWD applied to a stainless steel surface, dried for 24 hours and then swabbed (FIG. 8C pooled). CWD was extracted a 2^{nd} time from the same swabs to determine if additional RT-QuIC seeding activity could be recovered. The RT-QuIC seeding activity from a 2^{nd} extraction of CWD prions directly applied to the swabs (FIG. 8B) or recovered from surface (FIG. 8C) was decreased by 1 log compared to the first extraction (FIG. 9). Consistent results were observed for both the 1^{st} (FIG. 10) and 2^{nd} (FIG. 11) extracts in the RAF analysis. The extracts of negative controls which were DPBS-loaded swabs and uncontaminated surfaces did not initiate RT-QuIC reactions (FIG. 12). Overall, RT-QuIC detection of CWD prion recovered from stainless steel surface by swab sampling was comparable to detection of CWD prion when directly added to the RT-QuIC reactions.

Together, these results demonstrate that methods of prion sampling and recovery, in combination with ultrasensitive detection methods, can allow for highly sensitive prion detection from contaminated environmental surfaces.

### Example 3: RT-QuIC Detection CWD and TSEs Swabbed from Stainless Steel Surfaces

This Example demonstrates that other prions, in addition to CWD, and other prion-like polypeptides can be detected by RT-QuIC. This Example also demonstrates that RT-QuIC can successfully detect misfolded polypeptides with varied sonication and concentration conditions.

### Materials and Methods

To examine whether different sonication and concentration conditions can be used in RT-QuIC, the following experiments were performed.

Synthetic alpha-synuclein and brain homogenate (10% w/v) affected with four prion strains including sheep scrapie, transmissible mink encephalopathy (TME), cattle-adapted TME, or squirrel monkey-adapted CWD prions were diluted with PBS to desired levels to contaminate stainless steel surface (Table 4). CWD 307 CL and PBS were tested as positive and negative controls for extraction and detection. 50 μL of sample from each dilution was added to stainless steel surface in triplicates and dried at room temperature for 24 hours. Foam-tipped swabs were dampened with ultrapure water to swab surface. Swabs were either placed into 15 mL conical tubes (one swab per tube), preloaded with 500 μL of PBS, or handle-cut to fit into 1.7 mL microcentrifuge tubes (one swab per tube) preloaded with 300 μL of PBS, and then samples were extracted and concentrated under tested conditions (Table 5). Each swab was extracted for one time. Extracts from 15 mL tubes were transferred to 1.7 mL microcentrifuge tubes for concentration. For vacuum-concentrated samples, 50 μL of 0.1% SDS in PBS supplemented with N-2 (1×) were added for resuspension. For centrifuge-concentrated samples, supernatant was collected by pipet and the invisible pellet was resuspended in either 50 μL (307 CL) or 20 μL (synthetic alpha-synuclein) of 0.1% SDS in PBS. After resuspension, samples were either directly loaded to RT-QuIC plate or further diluted for detection.

TABLE 4
Materials and equipment used for swab extraction and concentration.

| | MNPRO | Creighton University |
|---|---|---|
| | Materials | |
| Samples | Synthetic alpha-synuclein, 307 | Scrapie, cattle adapted TME, |
| | CL (positive control) | TME, Squirrel Monkey |
| | | 307 CL (positive control) |
| Swab | Foam-tipped swab (Fisher Scientific) | |
| Stainless Steel plate | 316 L | 316 L |
| Tubes | 15 mL conical centrifuge tubes, 1.7 mL microcentrifuge tubes | |
| | Equipment | |
| Sonication | Qsonica Q700 with cup horn | Qsonica Q700 with cup horn |
| | | and microplate horn |
| Concentration | Centrifuge | Centrifuge |
| | | Vacuum concentration |
| | | (SpeedVac SPD1030) |

### Results

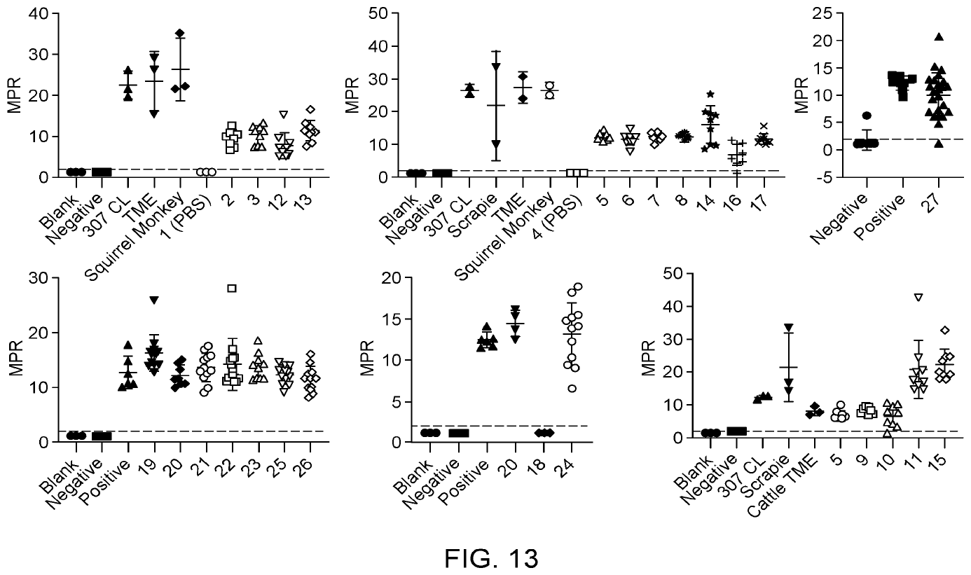

All samples at tested conditions were successfully detected, indicating a range of ultrasonic intensities from around 0.72 to around 8.84 watts/cm² are capable of extracting various misfolded polypeptides swabbed from stainless steel surface with both vacuum concentration and centrifuge at 16000× g or greater. Both microcentrifuge and centrifuge tubes can be used for swab extraction. RT-QuIC detection of misfolded polypeptides swabbed from stainless steel surface as maxpoint ratio (MPR) are shown in FIG. 13.

TABLE 5
Sonication and concentration conditions for swabs.

| Sample | | Dilution | | Ultrasonic Intensity | | Concentration |
|---|---|---|---|---|---|---|
| ID | Sample | (log) | Sonication | (Watts/cm²) | Tube | method |
| 1 | PBS | — | Microplate horn | ~2.15 | 1.7 mL | Vacuum |
| | | | ~300 watts | | | |
| 2 | 307 CL | −3 | Microplate horn | ~0.72 | 1.7 mL | Vacuum |
| | | | ~100 watts | | | |
| 3 | 307 CL | −3 | Microplate horn | ~2.15 | 1.7 mL | Vacuum |
| | | | ~300 watts | | | |
| 4 | PBS | — | Cup horn | ~8.84 | 1.7 mL | Vacuum |
| | | | ~280 watts | | | |
| 5 | 307 CL | −3 | Cup horn | ~3.79 | 1.7 mL | Vacuum |
| | | | ~120 watts | | | |
| 6 | 307 CL | −3 | Cup horn | ~8.84 | 1.7 mL | Vacuum |
| | | | ~280 watts | | | |
| 7 | 307 CL | −3 | Cup horn | ~1.58 | 15 mL | Vacuum |
| | | | ~60 watts | | | |
| 8 | 307 CL | −3 | Cup horn | ~8.84 | 15 mL | Vacuum |
| | | | ~280 watts | | | |
| 9 | 307 CL | −3 | Microplate horn | ~1.25 | 1.7 mL | Vacuum |
| | | | ~175 watts | | | |
| 10 | Scrapie | −2 | Microplate horn | ~1.25 | 1.7 mL | Vacuum |
| 11 | Cattle^{a} | −3 | ~175 watts | | | |
| 12 | TME | −3 | | | | |
| 13 | Sq.Monk.^{b} | −3 | | | | |
| 14 | Scrapie | −2 | Cup horn | ~3.79 | 1.7 mL | Vacuum |
| 15 | Cattle^{a} | −3 | ~120 watts | | | |
| 16 | TME | −3 | | | | |
| 17 | Sq.Monk.^{b} | −3 | | | | |
| 18 | PBS | — | Cup horn | ~6.63 | 1.7 mL | Centrifuge |
| | | | ~210 watts | | | 21000 × g |
| 19 | 307 CL | −3 | Cup horn | ~1.58 | 1.7 mL | Centrifuge |
| | | | ~50 watts | | | 21000 × g |
| 20 | 307 CL | −3 | Cup horn | ~1.58 | 1.7 mL | Centrifuge |
| | | | ~50 watts | | | 16000 × g |
| 21 | 307 CL | −3 | Cup horn | ~6.63 | 1.7 mL | Centrifuge |
| | | | ~210 watts | | | 21000 × g |
| 22 | 307 CL | −3 | Cup horn | ~6.63 | 1.7 mL | Centrifuge |
| | | | ~210 watts | | | 16000 × g |
| 23 | 307 CL | −3 | Cup horn | ~1.58 | 15 mL | Centrifuge |
| | | | ~50 watts | | | 21000 × g |
| 24 | 307 CL | −3 | Cup horn | ~1.58 | 15 mL | Centrifuge |
| | | | ~50 watts | | | 16000 × g |
| 25 | 307 CL | −3 | Cup horn | ~6.63 | 15 mL | Centrifuge |
| | | | ~210 watts | | | 21000 × g |
| 26 | 307 CL | −3 | Cup horn | ~6.63 | 15 mL | Centrifuge |
| | | | ~210 watts | | | 16000 × g |
| 27 | alpha-synuclein | −2 | Cup horn | ~1.58 | 1.7 mL | Centrifuge |
| | | | ~50 watts | | | 21000 × g |

^{a}Cattle-adapted TME;
^{b}Squirrel monkey-adapted CWD.
Note:
Microplate horn area: 139.67 cm²; Cup horn area: 31.67 cm². SpeedVac settings: 65° C., 2 hours or as needed to dry, 10 Torr vacuum. Centrifuge settings: 30 minutes, 4° C.

### Example 4: Herd-Level Surveillance of CWD in the Environment

Feeding devices having at least one sample collection surface (e.g., stainless steel sample collection surface) are placed in areas (e.g., specific geographic regions and/or farms) known to be populated by cervid herds. Exemplary feeding devices are shown in FIG. 6 and FIG. 7.

When feeding from a feeding device having at least one sample collection surface (e.g., stainless steel sample collection surface), a cervid leaves saliva and/or mucus deposits on the sample collection surface(s). A sample collection surface (e.g., stainless steel sample collection surface) of the feeding device is examined for the presence or absence of misfolded polypeptides (e.g., Prp^{Sc}) in the deposited saliva and/or mucus. For example, a sample collection surface (e.g., stainless steel sample collection surface) of a feeding device is swabbed with a foam swab and/or a cotton swab. Optionally, the swab is maintained in moist conditions (e.g., in a buffer solution such as a PBS solution). Polypeptides are extracted from a swab as described in Example 1. Misfolded polypeptides (e.g., Prp^{Sc}), if present, are detected and quantified as described in Example 1.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for assessing a population of non-human mammals for a prion disease, said method comprising:
swabbing a sample collection surface of a feeding device to obtain a swab comprising a sample, wherein said feeding device comprises a reservoir and a sample collection surface, wherein said reservoir contains food, and wherein said food can pass from said reservoir to said sample collection surface;
extracting polypeptides from said sample to obtain an extract;
concentrating said extract to obtain a concentrated extract;
detecting a presence of a misfolded polypeptide in said concentrated extract;
identifying said population of non-human mammals as having said prion disease if said presence of said misfolded polypeptide is detected; and
identifying said population of non-human mammals as not having said prion disease if said presence of said misfolded polypeptide is not detected.

2. The method of claim 1, wherein said sample collection surface is a material selected from the group consisting of stainless steel, mica, slate, aluminum, ceramic, and glass.

3. The method of claim 1, wherein said food is selected from the group consisting of corn, soybeans, oats, and commercial feed pellets.

4. The method of claim 1, wherein said sample is selected from the group consisting of saliva, mucus, and tongue epithelial cells.

5. The method of claim 1, wherein said non-human mammal is selected from the group consisting of elk, fallow deer, marsh deer, mule deer, muntjac, moose, pampas deer, red deer, reindeer, roe deer, sambar deer, sika, white-tailed deer, antelope, goats, camels, mink, cats, cows, sheep, mice, rats, hamsters, brocket, chital, macaques, lemurs, spider monkeys, and chimpanzees.

6. The method of claim 1, wherein said feeding device is located in a wilderness area.

7. The method of claim 1, wherein said feeding device is located in an urban setting, a suburban setting, or a rural setting.

8. The method of claim 6, wherein said feeding device is unattended by a human for from about 1 to about 14 days.

9. The method of claim 1, wherein said feeding device is located on a farm.

10. The method of claim 9, wherein said sample is obtained daily.

11. The method of claim 9, wherein said sample is obtained weekly.

12. The method of claim 1, wherein said swab is a cotton swab or a foam swab.

13. The method of claim 12, wherein said swab is stored in a solution prior to said extracting polypeptides from said sample to obtain said extract.

14. The method of claim 13, wherein said solution is a phosphate-buffered saline (PBS) solution.

15. The method of claim 13, wherein said swab is stored at a temperature of from about −80° C. to about 4° C.

16. The method of claim 1, wherein said extracting comprises sonication.

17. The method of claim 1, wherein said concentrating step comprises vacuum concentration.

18. The method of claim 1, wherein said misfolded polypeptide is detected using real-time quaking-induced conversion (RT-QuIC), enzyme-linked immunosorbent assay (ELISA), immunohistochemistry (IHC), protein misfolding cyclic amplification (PMCA), or western blotting.

19. The method of claim 1, wherein said misfolded polypeptide is selected from the group consisting of a misfolded polypeptide associated with scrapie (Prp^{Se}), a misfolded polypeptide associated with chronic wasting disease (PrP^{CWD}), a misfolded polypeptide associated with bovine spongiform encephalopathy (PrP^{BSE}), a misfolded polypeptide associated with Creutzfeldt-Jakob Disease (PrP^{CJD}), a misfolded polypeptide associated with feline spongiform encephalopathy (PrP^{FSE}), a misfolded polypeptide associated with transmissible mink encephalopathy (PrP^{TME}), and a misfolded polypeptide associated with camel spongiform encephalopathy (PrP^{CSE}).

20. The method of claim 1, wherein said prion disease is selected from the group consisting of chronic wasting disease (CWD), transmissible mink encephalopathy (TME), bovine spongiform encephalopathy (BSE), scrapie, feline spongiform encephalopathy, ungulate spongiform encephalopathy, and camilid spongiform encephalopathy.

21. A method for detecting a misfolded polypeptide, said method comprising:
swabbing a sample collection surface to obtain a swab comprising a sample;
extracting polypeptides from said sample to obtain an extract;
concentrating said extract to obtain a concentrated extract; and
detecting a presence of said misfolded polypeptide in said concentrated extract.

22. The method of claim 21, wherein said sample collection surface is a material selected from the group consisting of stainless steel, mica, slate, aluminum, ceramic, and glass.

23. The method of claim 21, wherein said collection surface is in a food processing facility, a water-treatment facility, or a hospital.

24. The method of claim 21, wherein said sample is selected from the group consisting of blood, urine, feces, saliva, and mucus.

25. The method of claim 21, wherein said sample is obtained daily.

26. The method of claim 21, wherein said sample is obtained weekly.

27. The method of claim 21, wherein said swab is a cotton swab or a foam swab.

28. The method of claim 27, wherein said swab is stored in a solution prior to said extracting polypeptides from said sample to obtain said extract.

29. The method of claim 28, wherein said solution is a PBS solution.

30. The method of claim 28, wherein said swab is stored at a temperature of from about −80° C. to about 4° C.

31. The method of claim 21, wherein said extracting comprises sonication.

32. The method of claim 21, wherein said concentrating step comprises vacuum concentration.

33. The method of claim 21, wherein said misfolded polypeptide is detected using RT-QuIC, ELISA, IHC, PMCA, or western blotting.

34. The method of claim 21, wherein said misfolded polypeptide is selected from the group consisting of a misfolded tau polypeptide, a misfolded alpha-synuclein polypeptide, and a misfolded amyloid beta polypeptide.

35. The method of claim 21, wherein said misfolded polypeptide is associated with a prion disease.

36. The method of claim 35, wherein said prion disease is selected from the group consisting of Creutzfeldt-Jakob disease (CJD), Gerstmann-Straussler-Scheinker disease (GSS), fatal familial insomnia (FFI), Alzheimer's disease, and Parkinson's disease.
